(19) United States
(12) Patent Application Publication
(10) Pub. No.: US 2020/0197566 A1
(43) Pub. Date: Jun. 25, 2020

(54) DIRECTED PLASMA NANOSYNTHESIS (DPNS) METHODS, USES AND SYSTEMS

(71) Applicant: The Board of Trustees of the University of Illinois, Urbana, IL (US)

(72) Inventors: Jean Paul ALLAIN, Champaign, IL (US); Zachariah KOYN, Champaign, IL (US); Brandon J. HOLYBEE, Champaign, IL (US)

(73) Assignee: The Board of Trustees of the University of Illinois, Urbana, IL (US)

(21) Appl. No.: 16/500,574

(22) PCT Filed: Apr. 6, 2018
(86) PCT No.: PCT/US18/26578

§ 371 Date: Oct. 3, 2019

Related U.S. Application Data
(60) Provisional application No. 62/483,105, filed on Apr. 7, 2017.
(60) Provisional application No. 62/483,074, filed on Apr. 7, 2017.
(60) Provisional application No. 62/556,048, filed on Sep. 8, 2017.
(60) Provisional application No. 62/556,120, filed on Sep. 8, 2017.

(51) Int. Cl. A61L 27/32; C23C 14/34; C23C 14/46; A61K 6/84; A61L 27/34

(52) U.S. Cl. CPC A61L 27/32; C23C 14/3442; C23C 14/46; A61L 2400/18; A61L 27/34; A61L 2420/02; A61L 2430/12; A61K 6/84

(57) ABSTRACT

Provided herein are systems and methods for the controlled surface modification of a material substrate, including, for example, generation of nanostructures, crystallographic or morphologic alterations and the removal of defects, changes in chemical composition and bond structure and the creation of thermodynamic metastable states. The provided systems and methods utilize one or more directed energetic particle beams with independently controlled parameters (e.g. incident angle, fluence, flux, energy, species, etc.) to precisely and efficiently generate enhanced surface properties beyond those of conventional plasma kinetic roughening.

### CROSS-REFERENCE TO RELATED APPLICATIONS

This application claims the benefit of and priority to U.S. Provisional Application No. 62/483,105, filed Apr. 7, 2017; U.S. Provisional Application No. 62/483,074, filed Apr. 7, 2017; U.S. Provisional Application No. 62/556,120, filed Sep. 8, 2017 and U.S. Provisional Application No. 62/556,048, filed Sep. 8, 2017, which are each hereby incorporated in their entirety to the extent not inconsistent herewith.

### BACKGROUND OF INVENTION

Each year millions of Americans undergo procedures involving medical implants, including hip replacements, spinal cord surgery, dental implants and arterial stents. However, there is significant risk involved in these procedures. Nearly one million people suffer from infections each year in the United States alone due to medical implants. One of the most critical challenges to the success of the implant is the interface between the implant material and the biological tissue it will replace. The direction of biomedical implant technology is the advent of smart (personalized), nutrient (bioresorbable) and low-cost biomaterials that are tailored to a patient's physiology. This likely involves design and synthesis of biomaterials for nutrient bioresorbable implants, ones that can dissolve over time in the body (with nutrients to accelerate healing) while the tissue is locally reconstructed (e.g. bone or artery reconstruction). One increasing challenge with achieving next-generation biomedical implants for bone or vascular reconstruction is the lack of a low-cost, non-toxic, scalable synthesis technology that enables high-fidelity design of bioactive interfaces that yield effective tissue reconstruction and prevent serious infection and integration issues. For example, an increasing health concern with peri-implantitis and infection in dental implants integrated to bone (Zarb 2013).

Controlled nanostructuring of material surfaces can dramatically increase functional properties, including increased bioactivity for medical implants and devices. Currently, some medical implants and devices receive some surface enhancement to increase bioactivity, such as osseointegration, osseoconduction, hydrophobicity, hydrophilicity, cell adhesion, etc. Conventional methods for material surface treatments include electrophoretic deposition, anodization, electrolysis, reactive DC magnetron sputtering, RF plasma sputtering, and x-ray sintering among others. However, many methods generate an interface between the surface coating and the substrate and fail to provide precise control of the surface modifying mechanism. Further, known methods are cost prohibitive due to chemical and energy requirements and/or the amount of time required to modify large surface areas.

Recent advances in material nanosurfacing include US Patent Publication 2015/0292077 which provides irradiation to a substrate surface in conjunction with a thermal beam for the fabrication of nanostructures. Further, this reference reports processes for control and selectivity of the nanostructures generated.

Gas cluster ion beams have also been proposed for generation of surface nanostructures for increasing the biofunctionality of a substrate. U.S. Pat. No. 9,144,627 relates to using accelerated gas cluster ion beams to modify surfaces of substrates in order to achieved enhanced biofunctionality.

It can be seen from the foregoing, that there remains a need in the art for the precise generation of nanostructures on material substrates, including methods for the modification of larger surface areas on complex shaped surfaces. Methods to modify multiple surface properties, including the generation of different nanostructures upon a single surfaces, modifications to chemical composition or bond properties, and/or alteration of the surface crystallographic structure would be further beneficial in generating new materials with enhanced surface properties, including biomaterials with multiple increases or changes to bioactivity not achievable by known methods or processes.

### SUMMARY OF THE INVENTION

Provided herein are systems and methods for the selective modification of the structure and/or properties of a surface of a substrate, for example, providing nanoscale control of surface topography, topology, morphology, crystal structure and/or composition. In embodiments, for example, the present methods and systems provide selective fabrication of nanostructures, crystallographic or morphologic alterations and/or the removal of defects, changes in chemical composition and bond structure and the creation of thermodynamic metastable states, through use of directed plasma nanosynthesis (DPNS).

Systems and methods of certain embodiments provide a directed energetic particle beam(s) with independently controllable parameters (e.g., incident angle, fluence, flux, energy, species, extraction voltage, etc.) to engineer surface structures and properties beyond those of conventional techniques, such as plasma kinetic roughening. In embodiments, multiple and complementary surface properties are selectively modified simultaneously, for example, to achieve a selected functionality such as biological activity (e.g., anti-bacterial, anti-inflammatory, osteointegrative, etc.) and/or enhanced physical property (e.g., hydrophilicity, hydrophobicity, bioresorption, etc.). The methods include plasma-based approaches for efficient selection, extraction and combination of ions to achieve ion beam exposure conditions useful for a range of application, for example, via large area and/or multiplexed ion beams. Further, methods and systems of certain aspects provide ion beam exposure with a selected three dimensional spatial profile, for example, a selected three dimensional spatial profile complimentary to a non-planer substrate surface, such as surfaces of an implantable material and/or medical device.

The systems and methods of certain embodiments are useful for enhancing mechanical and/or physical properties (e.g., elasticity, modulus, surface texture, porosity, wettability, surface tension, hydrophilicity, hydrophobicity, etc.) of a substrate. In embodiments, the described systems and methods are useful for modifying medical implants and devices for enhanced bioactivity, for example, providing enhanced osseointegration, osseoconductivity, cell adhesion, hydrophobicity, hydrophilicity, resorption, anti-bacterial properties, anti-inflammatory properties and/or drug delivery capabilities. The systems and methods provided herein are versatile and, thus, applicable a wide range of substrate materials (e.g., metals, alloys, ceramics, natural polymers and synthetic polymers) and surface shapes (e.g., planar, contoured, curved, etc.).

In an aspect, provided is a method of generating structures on a substrate, the method comprising: i) providing the substrate having a substrate surface; ii) generating a first directed energetic particle beam from a low temperature plasma source; and iii) directing the first directed energetic particle beam onto the substrate surface, thereby generating the structures on the surface; wherein the first directed energetic particle beam has one or more beam properties selected to generate the structures characterized by one or more nanoscale features. In an embodiment, the nanoscale topography is selected from the group consisting of hexagonal ordered arrays, square ordered arrays, square ordered arrays, and combinations thereof.

In an aspect, provided is a method of generating structures on a substrate, the method comprising: i) providing the substrate having a substrate surface; ii) generating a first directed energetic particle beam and a second directed energetic particle beam from a low temperature plasma source; and iii) directing the directed energetic particle beams onto the substrate surface, thereby generating the structures on the surface; wherein the first directed energetic particle beam has one or more first beam properties and the second directed energetic particle beam has one or more second beam properties different from said first beam properties, wherein said first and second beam properties are selected to generate the structures characterized by one or more nanoscale features.

In embodiments, for example, the nanoscale features are a preselected nanoscale composition, morphology, topology, topography, crystal structure, density of defects, charge density, bond hybridization or any combination thereof. In an embodiment, the nanoscale morphology is selected from the group consisting of nanorods, nanocones, nanowalls, nanoripples, pores, and combinations thereof. In an embodiment, the nanoscale features correspond to a thermodynamically metastable state.

In embodiments, the one or more beam properties is intensity, fluence, energy, local flux, incident angle, mass, species, momentum, charge state or any combinations thereof. In embodiments, for example, the local fluence is between about 10¹²-10²⁰ ions/cm², or the energy is between about 50-5000 eV, or the local flux is between about 10¹²-10¹⁶ ions/cm²/s, or the incident angle is between about 0-90°, or the mass is between about 1-131 amu, or the species is selected from the group consisting of H, He, O₂, N₂, Ne, Ar, Kr, Xe, and combinations thereof, or the momentum is between about 10⁻²⁴-10⁻²⁰ kg*m/s, or the charge state is ±1, 2, 3.

In an embodiment, for example, the directed energetic particle beam comprises one or more ions, electrons, neutrals, free radicals or combinations thereof. In embodiments, the one or more beam properties are the ion composition, neutral composition, free radical composition, the ratio of ion abundance to neutral abundance or any combination of these.

In embodiments, the directed energetic particle beam is incident upon the substrate surface from a plurality of directions, for example characterized by a distribution of directions. In embodiments, the plurality of directions of the directed energetic particle beam is achieved by a porous and non-planar electromagnetic grid in fluid communication with the plasma. In an embodiment, the porous and non-planar electromagnetic grid is a cylindrical electromagnetic grid. In embodiments, for example, the directed energetic particle beam is incident upon a non-planar inner surface of the substrate, or, is incident upon a non-planar outer surface of the substrate.

Additional energetic particle beams may be incorporated to simultaneously provide multiple surface modifications. The additional beams may be independently controlled in terms of type of beam or various beam parameters do provide greater control of surface modification and allow for multiple and simultaneous nanoscale modifications.

In embodiments, the provided method further comprises directing one or more additional beams onto the substrate surface, wherein the addition beams are one or more particle beams, radiation beams or a combination thereof. In an embodiment, for example, the one or more additional beams are characterized by at least one beam property that differs from the one or more beam properties of the directed energetic particle beam. In an embodiment, the one or more additional beams are directed energetic particle beams. In an embodiment, the one or more additional beams provides a nanoscale feature which is modified as compared to a nanoscale feature which is provided by use of a single beam. In embodiments, the one or more additional beams is a focused ion beam, a broad ion beam, a thermal beam, a plasma generated beam, an optical beam or any combination of these. The use of energetic particle beams allows for substrate quench rates that are greater than traditional thermal and/or chemical processing methods. Quench rates may, for example, be nearly instantaneous as the directed particle beams may athermally interact with the substrate. In embodiments, the provided energetic particle beams provide a quench rate selected from the range of 10¹¹ K/s to 10¹⁴ K/s (degrees Kelvin per second). In embodiments, for example, the substrate is quenched in less than or equal to 10 μs, or optionally, less than or equal to 1 ns.

In an embodiment, the provided method further comprises providing one or more additional reactive species or surfactants at a point of contact between the energetic particle beams and the substrate.

In embodiments, for example, the substrate is a metal, an alloy, a ceramic, a polymer, a glass, a tissue or any combination of these. In an embodiment, the substrate surface is the surface of a medical device, an implant, a tissue, a scaffold, a syringe, a needle, a scalpel, a surgical rod, a surgical plate, a surgical screw or any combination of these.

The provided systems and methods may include the deposition of material (e.g. via sputtering) upon the surface of the substrate, allowing for alterations of chemical composition, the creation of alloys or modifications of the interaction between the beam(s) and the substrate, for example, to promote the generation of nanostructures.

In an embodiment, the provided method further comprises depositing one or more agents on the surface of the substrate. In embodiments, the step of depositing the one or more agents is performed by sputtering a target in communication with the directed energetic particle beam. In embodiments, for example, the agents are selected from the group comprising: metals, metal oxides, polymers, glasses, ceramics, tissues, pharmaceuticals, surfactants and combinations thereof.

In an aspect, provided is a system for generating a three dimensional energetic particle beam comprising: i) a low temperature plasma source for generating ions; and ii) at least one porous and non-planar electromagnetic grid in fluid communication with the plasma, wherein the electromagnetic grid accelerates the ions to generate a directed energetic particle beam having one or more selected beam properties. A wide range of non-planar geometries are useful for electromagnetic grids including contoured (convex, concave, or combinations), round, cylindrical, spherical, conical, annular, and segmented. In some embodiments, the non-planar geometry of the electromagnetic grid has a shape that is complementary to the shape of the substrate undergoing processing, for example, having a geometry allowing for insertion of the electromagnetic grid into or throughout a feature of the substrate, such as an aperture, opening or channel. A wide range of porosities are useful for electromagnetic grids including a porosity selected from the range of 0.1 mm to 5 mm, and optionally for some applications 1 mm to 3 mm.

In embodiments, for example, the electromagnetic grid has a cylindrical shape with an open top surface, an open bottom surface and a porous axial surface. In an embodiment, the system further comprises: a) a second porous and non-planar electromagnetic grid in fluid communication with the plasma; and b) an agent in communication with said at least one focused ion beam.

In an embodiment, the low temperature plasma source comprises a waveguide operationally connected to a power source, wherein the power source is selected from the group consisting of a dissipated radio frequency, a microwave energy selected from the range of about 10 and 1000 Watts, and a high voltage of selected from the range of 100 and 10,000 V, applied to two or more electrodes. In embodiments, the provided system further comprises a matching network to minimize the reflected power between the power supply and the plasma due to plasma impedance. In embodiments, for example, a voltage selected from the range of 100 to 5000 V applied to the at least one porous and non-planar electromagnetic grid to control the absolute and relative ratio fluxes and the acceleration of particles within a directed energetic particle beam.

In an embodiment, the at least one porous and non-planar electromagnetic grid maintains a constant voltage potential during operation of the system. In embodiments, the at least one porous and non-planar electromagnetic grid is electrically and physically isolated from the power source. In embodiments, for example, the at least one porous and non-planar electromagnetic grids is mounted using ceramic discs, rings, or cylinders.

As noted herein, the described methods may provide compositions that may be rendered anti-bacterial. On many surfaces exposed to the environment, there is the risk that a microbial biofilm may form on a surface. The compositions of the invention may be used together with any surface. The surface is not limited and includes any surface on which a microorganism may occur, particularly a surface exposed to water or moisture. Treating surfaces to avoid films of antimicrobial compounds or manufacturing with them the working surfaces of laboratories (clinical, microbiological, water analysis, food), of businesses handling fresh food (butchers, fishmongers, etc.), of hospital buildings and health centers, to mention just a few examples, guarantees the suitable hygienic conditions for development of the work and eliminates the risk of contamination and infections.

Such inanimate surfaces exposed to microbial contact or contamination include in particular any part of: food or drink processing, preparation, storage or dispensing machinery or equipment, air conditioning apparatus, industrial machinery, e.g. in chemical or biotechnological processing plants, storage tanks and medical or surgical equipment. Any apparatus or equipment for carrying or transporting or delivering materials, which may be exposed to water or moisture is susceptible to biofilm formation. Such surfaces will include particularly pipes (which term is used broadly herein to include any conduit or line). Representative inanimate or abiotic surfaces include, but are not limited to food processing, storage, dispensing or preparation equipment or surfaces, tanks, conveyors, floors, drains, coolers, freezers, equipment surfaces, walls, valves, belts, pipes, air conditioning conduits, cooling apparatus, food or drink dispensing lines, heat exchangers, boat hulls or any part of a boat's structure that is exposed to water, dental waterlines, oil drilling conduits, contact lenses and storage cases. As noted above, medical or surgical equipment or devices represent a particular class of surface on which a biofilm may form. This may include any kind of line, including catheters (e.g. central venous and urinary catheters), prosthetic devices e.g., heart valves, artificial joints, false teeth, dental crowns, dental caps and soft tissue implants (e.g. breast, buttock and lip implants). Any kind of implantable (or “in-dwelling”) medical device is included (e.g. stents, intrauterine devices, pacemakers, intubation tubes, prostheses or prosthetic devices, lines or catheters). An “in-dwelling” medical device may include a device in which any part of it is contained within the body, i.e. the device may be wholly or partly in-dwelling. Plastic materials with antimicrobial properties can also be used in manufacturing handles, handlebars, handgrips and armrests of public transport elements, in rails and support points in places widely used, in the manufacturing of sanitary ware for public and mass use, as well as in headphones and microphones of telephones and audio systems in public places; kitchen utensils and food transport, all with the purpose of reducing the risk of propagation of infections and diseases.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. Inward-facing DPNS source.

FIG. 2. Outward-facing DPNS source.

FIG. 3. Cylindrical grid design.

FIG. 4. Potential waveguide designs for microwave plasma generation.

FIG. 5. Microplasma source for treating complex surface geometries.

FIG. 6. Plasma SEED grid design.

FIG. 7. Flow chart of an exemplary method for surface modification.

FIG. 8A shows long nano-walls, gas Kr (MAG ×80,000).

FIG. 8B shows short nano-walls, gas Kr (MAG ×80,000).

FIG. 9A shows narrow nano-cones, gas Kr (MAG ×50,000).

FIG. 9B shows wide nano-cones, gas Kr (MAG ×50,000).

FIG. 10 shows nano-ripples, gas Kr (MAG ×120,000).

FIG. 11 shows nano-walls, gas Ar (MAG ×30,000).

FIG. 12A shows narrow nano-cones, gas Ar (resol: 500 nm)

FIG. 12B shows wide nano-cones, gas Ar (resol: 500 nm).

FIG. 13 shows round plate formation, gas Ar (resol:1 micron).

FIG. 14 showing nanowalls, fluence 7.5×10¹⁷ (resol 1 micron)

FIG. 15A shows narrow cones, fluence 7.5×10¹⁷ (resol: 500 nm).

FIG. 15B shows wide cones, fluence 7.5×10¹⁷ (resol: 500 nm).

FIG. 16A shows long nano-walls, fluence 5×10¹⁷(resol: 500 nm).

FIG. 16B shows short nano-walls, fluence 5×10¹⁷ (resol: 500 nm).

FIG. 17A shows narrow nano-cones, fluence 5×10¹⁷ (resol: 500 nm).

FIG. 17B shows wide nano-cones, fluence 5×10¹⁷ (resol: 500 nm).

FIG. 18A shows small nano-walls with smooth surface, fluence 2.5×10¹⁷ (resol: 2 microns)

FIG. 18B shows small nano-walls, fluence 2.5×10¹⁷, at high resolution (resol: 500 nm))

FIG. 19A shows narrow nano-cones, fluence 2.5×10¹⁷ (resol: 500 nm).

FIG. 19B shows wide nano-cones, fluence 2.5×10¹⁷ (resol: 500 nm).

FIG. 20 show nano-wall formation at high resolution, fluence 1×10¹⁷ (resol: 500 nm).

FIG. 21, showing nano-cone formation at high resol, fluence 1×10¹⁷ (resol: 500 nm).

FIG. 22 shows SEM images showing the evolution of surface nano-patterning of Ti6Al4V samples for different incidence angles with Ar+ irradiation.

FIG. 23A shows surface characteristics of Ti6Al4V samples before DIS processing.

FIG. 23B shows surface characteristics of Ti6Al4V samples before DIS processing.

FIG. 24A shows survey scans for Ti alloy samples treated with a fluence of 1×10¹⁸ cgs for Ar irradiation at 60°.

FIG. 24B shows Al2p, C1s, N1s, O1s, Ti2p, and V2p for Ti alloy samples treated with a fluence of 1×10¹⁸ cgs for Ar irradiation at 60°.

FIG. 24C shows survey scans for Ti alloy samples.

FIG. 24D shows Al2p, C1s, N1s, O1s, Ti2p, and V2p for Ti alloy samples.

FIG. 25A shows survey scans for Ti alloy samples treated with a fluence of 7.5×10¹⁷cgs for Ar irradiation at 60°.

FIG. 25B shows Al2p, C1s, N1s, O1s, Ti2p, and V2p for Ti alloy samples treated with a fluence of 7.5×10¹⁷cgs for Ar irradiation at 60°.

FIG. 26A shows survey scans for Ti alloy samples treated with a fluence of 5×10¹⁷cgs for Ar irradiation at 60°.

FIG. 26B shows Al2p, C1s, N1s, O1s, Ti2p, and V2p for Ti alloy samples treated with a fluence of 5×10¹⁷cgs for Ar irradiation at 60°.

FIG. 27A shows survey scans for Ti alloy samples treated with a fluence of 2.5×10¹⁷cgs for Ar irradiation at 60°.

FIG. 28A shows survey scans for Ti alloy samples treated with a fluence of 1×10¹⁷cgs for Ar irradiation at 60°.

FIG. 28B shows Al2p, C1s, N1s, O1s, Ti2p, and V2p for Ti alloy samples treated with a fluence of 1×10¹⁷ cgs for Ar irradiation at 60°.

FIG. 29 shows an embodiment of a complete plasma source featuring mounting flange (bottom), three electrically isolated support mounts, RF coil to generate plasma, and dual isolated electromagnetic grids to control particle flux to the interior, supported by ceramic rings.

FIG. 30 shows an expanded view of RF coil generating plasma.

FIG. 31 shows an embodiment of the grid assembly, utilizing full cylindrical grids. Exploded view of grid assembly featuring inner grid (top) that controls particle energy, outer grid (bottom) that enhances particle extraction from plasma, and ceramic rings to secure grids in place.

FIG. 32 shows a design of an embodiment of an upper ceramic piece designed to separate and secure the grids.

FIG. 33 shows the design of a lower ceramic piece with apertures allowing support components of grids to pass through and connect to structural components.

FIG. 34 shows one embodiment of the grid assembly, constructed from two rectangular pieces, connected by screws, nuts, and ceramic washers shown, that curls into cylinders as constructed due to hole spacing offset.

FIG. 35 shows a constructed view of the grid assembly embodiment comprising two rectangular pieces, connected by screws, nuts, and ceramic washers shown, curled into cylinders due to hole spacing offset.

FIG. 36 shows an embodiment of grid assemblies utilizing complete cylinder grids, showing grids and mounting supports (ceramic cylinders not shown).

FIG. 37 provides a graph describing time on the x-axis and strength on the y-axis for various implant types.

FIG. 38 SEM images of middle part of a commercial titanium alloy dental implant. The studied area is polished Ttanium alloy (without SLA treatment) localized in the middle part. DPNS shows to be effective and develops small nanofeatures in the polished part. In addition, this complex topography which combines different planes, angles, pores does not suppose any obstacle to the development of nanoplatelets due to DPNS surface modification.

FIG. 39 SEM images of middle part of a commercial titanium alloy dental implant with the special focused on the SLA pretreated surface. The studied area is the SLA treated middle part in which DPNS shows to be effective as well and could develop small nanofeatures with similar morphology and size than in the previous polished area. This fact opens new frontiers in which complex medical devices can be improved without using chemical and toxic compounds.

FIG. 40 SEM images of lower part of commercial titanium alloy dental implants. The Argon ions of DPNS surface modification arrives at this area showing the presence of similar nanofeatures previously describe in FIGS. 38 and 39. Once more, DPNS shows to be effective in the surface modification of the 3D structures. In this case, the SLA modification of SLA does not create any obstacle modifying in deeper the dental implant surface at the nano-scale order.

FIG. 41 SEM images of the upper part of commercial titanium alloy dental implant. The studied area is polished Titanium alloy (without SLA treatment) after DPNS processing. Small nanostructures are presented in this area due to DPNS treatment.

FIG. 42 Mechanism of ion beam irradiation inside the pit of porous titanium samples by DPNS. Notice, the incidence of ion beam inside the pore and how it modifies the surface as well there is an energy deposition which subsequently continuous with the ion beam in other areas. This mechanism explains how the nanostructures are developed in other walls inside the pit even those that are not directly exposed to the ion beam.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Directed energetic particle beam,” as used herein, refers to a stream of accelerated particles generated from a low-energy plasma. In some embodiments, directed energetic particle beam is a focused or broad beam ion beams capable of delivering a controlled number of ions to a precise point or area upon a substrate over a specified time. Directed energetic particle beam may include ions and additional non-ionic particles including subatomic particles or neutral atoms or molecules. In embodiments, directed energetic particle beams provide individual ions to the target location. Examples of directed energetic particle beams include focused ion beams, broad ion beams, thermal beams, plasma generated beams, optical beams and radiation beams.

“Electromagnetic Device” refers to a device with an electromagnetic charge capable of accelerating ions or particles, including subatomic particles. In embodiments, for example, electromagnetic device refers to charged grids having an electromagnetic bias. In some embodiments, electromagnetic devices are a plurality of charged grids having multiple electromagnetic biases. In some embodiments, electromagnetic devices are three dimensional including cylinders, annuli, polygonal prisms, cones or spheres.

“Beam parameter” or “beam property” refers to a user or computer controlled property of beam, for example, an ion beam. Beam parameter may refer to incident angle with a target substrate, fluence, energy, beam composition and ion species. Beam parameters may be adjusted to provide selected interactions between the beam and the target substrate to generate specific nanostructures or enhance specific properties of the substrate. Beam parameters may be controlled by a variety of means, including adjustments to electromagnetic devices in communication with the beam, adjusting the gas or energy source used to generate the beam or physical positioning of the beam in reference to the target.

“Nanofeature” refers to nanostructures having relatively small dimensions generated on the surface of a material and changes in composition, crystal structure or bond hybridization on a surface. Nanostructures may refer to nanocolumns, nanoripples, nanopillars, nanorods, nanowires and/or quantum dots. Nanostructures include self-assembled nanostructures. In embodiments, for example, nanofeature refers to surface depths or structures generated on a surface having dimensions of less than 1 μm, less than or 100 nm, less than 50 nanometers, or in some embodiments, less than 10 nm. Nanofeatures may provide a material with a variety of beneficial characteristics including increased or decreased hydrophobicity (or conversely hydrophilicity), increased or decreased osseointegration, increased or decreased corrosion resistance, increased or decreased surface hardness and increased or decreased protein interaction.

“Agent” refers to a material in communication with the described beams which may be sputtered or dislodged from its original position and deposited on the surface of a substrate. In embodiments, agent refers to a sputterant deposited on a substrate through the process of ion beam deposition. Agent may refer to metals, metal oxides, and/or surfactants. Agents may provide the target substrate with enhanced surface properties or promote the self-assembly of nanostructures. Agents may also generate nanoscale alloy compositions between two metals at the surface of substrate.

“Substrate” refers to the target of an ion beam as described herein. In embodiments, substrates may comprise any material capable of forming nanostructures. Examples of substrates include metals, metal oxides, alloys, naturally occurring polymers, synthetic polymers and composite materials. In some embodiments, substrates may have three dimensional surfaces or multiple planar surfaces which interact with one or more ion beams simultaneously.

“Multiplexing” refers to simultaneously modifying the target substrate in more than one way, for example, by providing two or more directed particle beams at the substrate having different properties, for example, to generate or modify at least one nanoscale domain (e.g. nanoscale features, crystalline domains, compositional domains, distributions of defects, changes in bond hybridization. In some embodiments, for example, a single directed particle beam may have one or more beam properties to generate or modify multiple nanoscale domains on the substrate. In embodiments, multiple direction particle beams are generated from the same plasma source.

The technology as described in the present disclosure includes an advanced nanomanufacturing process as described herein, advanced tools particular for this process and a number of unique nano-scale structures generated as a result of the processing.

In one embodiment, provided is an atomic-scale additive nanomanufacturing process capable of transforming materials with multi-functional properties without the need for expensive heat cycles, toxic chemical processes or thermodynamic limitations of material compatibility in processing. The interface between plasma and material becomes an open thermodynamic system driven far from equilibrium by a rich variety of physical mechanisms, including high-energy kinetic disordering, compositional phase dynamics, and the emergence of metastable material states. The instabilities that arise due to these mechanisms lead to the evolution of well-ordered nanostructures, the compositional and morphological characteristics of which dictate the material properties.

Described is the use of directed energetic particle beams drawn from a low-temperature plasma (gas discharge) in a manner that controls the energy, species and intensity of the respective beams from the aforementioned plasma. The particles may be combined with additional reactive atoms and/or surfactants that interact with material surface inducing variation in a number of properties including: surface chemistry, composition, topography, topology, charge density and bond hybridization. In some cases the technology can manipulate these properties independently providing for multi-functionality on the material surface without modification to the bulk material.

The invention also consists of utilizing a 3D plasma source enabling the modification of complex 3D material structures (e.g. porous structures). When using surfactants the plasma source can be “seeded” with additional ultra-low energy reactive particle beams with the right energy and flux combination to tailor specific changes to the material surface (this mode is known as directed “soft” plasma nanosynthesis or DSPNS). Depending on material type the energetic particles are selected both in mass and species to result in the desired material property (e.g. hydrophobicity, anti-bacterial for biomaterials, etc . . . ). The material can be a polymer, metal, ceramic, or semiconductor and the synthesis can be done over large areas, at room temperature and over a short period of time (e.g. seconds).

The processes of the invention transform materials into “adaptive smart interfaces” tailored to respond uniquely to a pre-defined environment and adapt to its conditions. DPNS is designed to independently modify surface topography, composition and charge density yielding increase of surface energy and surface-to-volume ratios by factors of 50-100% and 100-1000, respectively.

DPNS and DSPNS are both processes of the invention that include a use of a plasma source enabling the modification of existing product materials (e.g. on a biomedical stent, implant device, etc . . . ) improving their properties or synthesizing completely new class of materials.

DSPNS enables a single source that addresses the problematic use of thin-film coatings for bioactive interfaces, which can potentially lead to osteolysis and chronic inflammation. Coating disintegration and delamination is also a prevalent problem that cannot be solved with current synthesis approaches that include: electrophoretic deposition, anodization, electrolysis, reactive DC magnetron sputtering, RF plasma sputtering, and x-ray sintering among others. One of the primary issues with these conventional technologies is the formation of the interface between the coating and biomedical material substrate.

Therefore, features of DPNS and DSPNS are: 1) low cost (e.g. they are a low-temperature process; heat cycles during synthesis make-up 30-40% of the current processing cost of surface modification techniques), 2) green and sustainable (does not require harsh chemicals for synthesis and can enhance usability of natural materials), and 3) scalable (particle irradiation can be conducted throughput levels of about 1012 micron²/hr or a modification of a 6-inch wafer in about 10 seconds). Another added benefit and potentially disruptive approach is the ability to modify a surface composition and chemistry independent of the topography with high-fidelity. In other words, inducing a surface that can potentially enhance cell adherence and proliferation while repelling bacteria, for example.

In one embodiment, the present invention provides a method of generating structures on a substrate. In this embodiment, a substrate having a substrate surface is provided and a directed energetic particle beam from a low temperature plasma source is generated, and this energetic particle beam is directed onto the substrate surface, thereby generating said structures on said surface; and where directed energetic particle beam has one or more beam properties selected to generate said structures characterized by one or more nanoscale features.

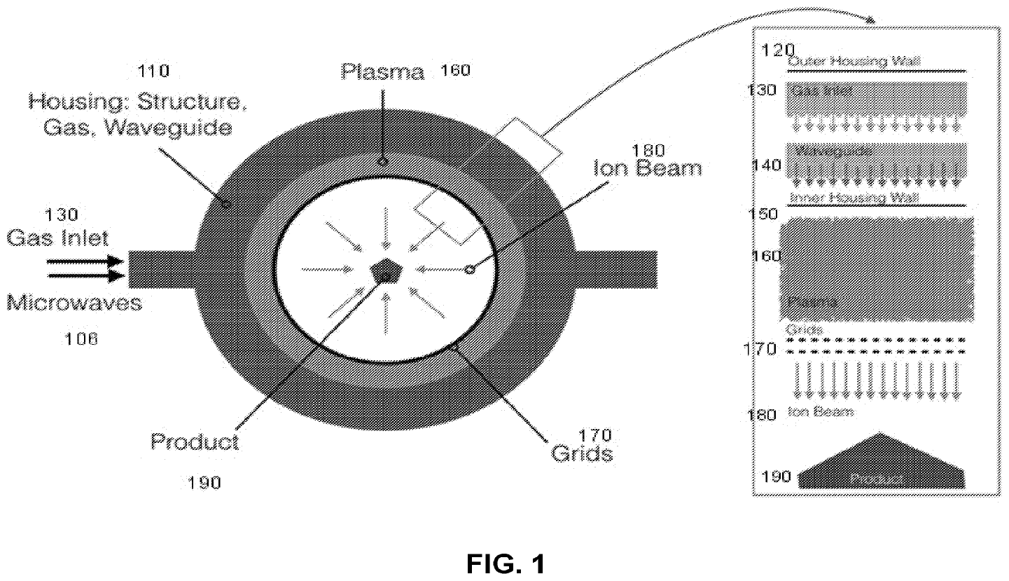

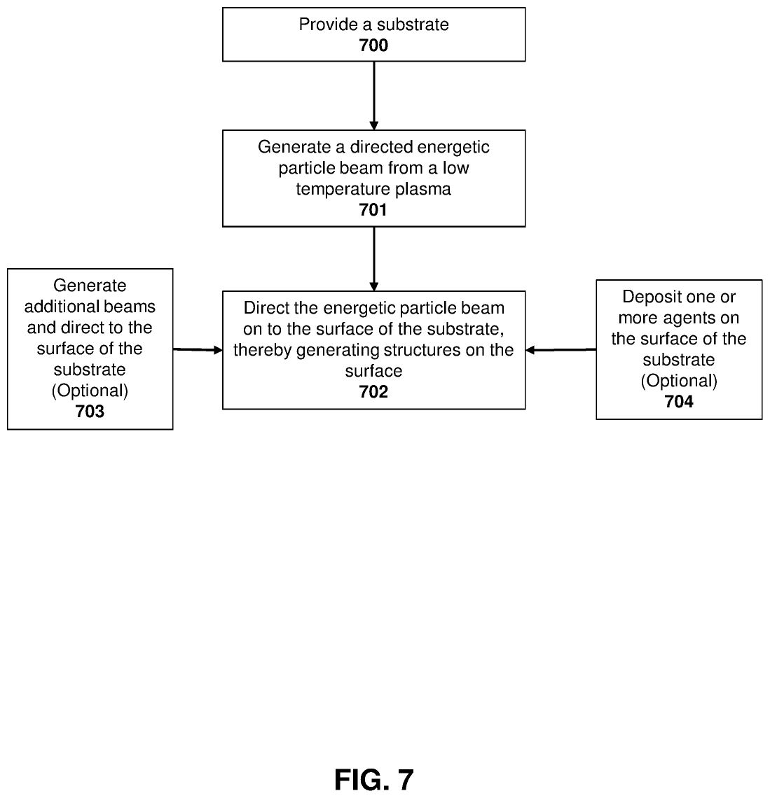

FIG. 7 shows a flow diagram of an exemplary embodiment of a DPNS that may be carried out by the arrangement of FIG. 1. In this embodiment, the DPNS produces nanostructures on the substrate surface 802. In first step 800, a substrate is provided in a fixture, not shown, where the directed energetic particle beam from a low temperature plasma 801 may operate on the substrate 800 with a surface. The directed energetic particle beam(s) from a low temperature plasma source are directed to the substrate surface in accordance with parameters and/or properties that correspond to a desired nanostructure topology. The parameter control may occur in an automated fashion, such as under the control of a numerical control device or special purpose computer, including a processing device and a memory containing programming instructions (not shown). In an optional step, additional beam(s) 803 may be generated and directed to the surface of the substrate 802 also in accordance with parameters and/or properties that correspond to a desired nanostructure topology. Optional step 804 includes depositing one or more agents on the surface of the substrate.

The methods of the invention for plasma processing provide the advantages of being scalable, versatile, and low-cost. Methods and devices to accomplish the aims of the invention are also called “Plasma 3D” herein. Traditional equipment in the materials processing and synthesis sector suffers from three limitations. Current ion beam sources are designed to treat flat, 2D surfaces and it is difficult or impossible to use them on complex structures. Similarly, plasma immersion can treat more complex structures, but suffers from a loss of the tight processing control that is possible with ion beams. The third and arguably more serious limitation of current technologies is the lack of high-fidelity control of specific particle beam fluxes, species and energies that independently can induce changes of both surface morphology and composition by careful variation of energy density deposition.

In embodiments, for example, the substrate is a material having at least one surface that is capable of being modified. A substrate may have one or more surfaces, such as an outer surface of a substrate or an inner surface of a substrate. The outer surface may be the exterior of the substrate. Alternatively, if the substrate has a cavity or hollow portion, the surface may be inside the cavity or hollow portion, and may be referred to as an inner surface. The substrate may be a metal, an alloy, a ceramic, a polymer, a semiconductor, a semi-metal, a non-crystalline metal, a pseudo-alloy, a composite or any combination of these. In one embodiment, the substrate is a medical device, dental or crown implant, joint prosthesis, spine implant, tissue scaffold, or other material as known in the art. Exemplary materials include, without limitation, titanium, titanium alloys, magnesium, natural polymers (chitosan, nanocellulose), nanoparticles (Ag, Au, FeO, ZnO, ZnS, carbon allotropes).

Methods of the invention include the step of generating directed energetic particle beam from a low temperature plasma source. Low temperature plasma generation methods and sources are known in the art, and may also be referred to as partially ionized plasmas (gas discharges). Plasma processing sources are known in the art, for example, Tectra GmbH Physikalische Instrumente (GENII PLASMA ION SOURCE) and Oxford Instruments (ISE 5 ion sputtering source). Also SVT Associates, Inc. provides the RF-6.02 Plasma Source. While the principles and methods for creating plasma sources are known, these plasma processing methods create only mono-directional particle beams, which limits their usage to flat, 2D surfaces, unlike the present methods utilizing schematics with variable geometry allowing more complex surfaces to be treated.

In some embodiments, low temperature plasmas useful for the present invention are gasiform plasmas with electron temperature under 10 eV, electron density typically from 10¹⁴ to 10²⁴ m⁻³. In general, low temperature plasmas have a low degree of ionization at low densities. This means the number of ions and electrons is much lower than the number of neutral particles (molecules). Different particles inside the plasma, i.e. neutrals, ions and electrons, can have different temperatures or energies. Indeed, in many applications, the background gas is near room temperature. In this regard, gas phase reaction activation energy can be driven by electron impact rather than thermally and the substrate is not subjected to extreme heating, which is useful for functionalizing temperature sensitive substrates such as polymers.

In embodiments, for example, the nanoscale features are a preselected nanoscale composition, morphology, topology, topography, crystal structure, density of defects, charge density, bond hybridization, surface energy or any combination thereof. In an embodiment, the nanoscale features correspond to a thermodynamically metastable state. Nanoscale features include nanoscale walls (e.g. parallel walls, non-parallel walls, etc.), nanocones, nano and micro-scale pillars, and nanoripples. These nanofeatures can include nanoscale parallel walls of, for example, about 3 nm to about 250 nm in height, or about 0.1 nm to about 1000 nm, about 0.5 nm to about 750 nm, about 1 nm to about 500 nm, about 2 nm to about 400 nm, about 3 nm to about 250 nm, about 5 nm to about 200 nm, about 10 nm to about 150 nm in height. These nanofeatures can also include nanocones of, for example, about 2 nm to100 nm in dimension with about 10 nm to 40 nm in length. Embodiments include dimensions of about 0.1 nm to about 500 nm, about 0.5 nm to about 400 nm, about 1 nm to about 200 nm, about 2 nm to about 100 nm. The nanofeatures can also include nanoripples of, for example, 50 nm thin and with lengths close to 0.1 nm to 0.5 nm.

In embodiments, the one or more beam properties is the gas, intensity, fluence, energy, flux, incident angle, species mass, charge, cluster size, molecule or any combinations thereof. In an embodiment, for example, the directed energetic particle beam comprises one or more ions, neutrals or combinations thereof. In embodiments, the one or more beam properties are the ion composition, neutral composition, the ratio of ion abundance to neutral abundance or any combination of these. In embodiments, the directed energetic particle beam is incident upon the substrate from a plurality of directions.

In one embodiment, nanostructures may be obtained as function of energetic particle species, fluence and incident angle with respect to the surface normal. For example, energetic particle species may include those obtained from gases such as Kr, Ar, Ne, Xe, H, He, O₂ and/or N₂. Fluence can be, for example, between 1×10¹⁷ to 1×10¹⁸particles per second per square meter, but may vary from 0.1×10¹⁷ to 50×10¹⁷. In some embodiments, fluence is 1×10¹⁷, 2.5×10¹⁷, 5×10¹⁷, or 1×10¹⁸ Finally, incident angle may be varied in single degrees between the angles of 0 and 80 degrees, in some embodiments, for example, 30 degrees, 60 degrees, and 80 degrees. In some embodiments, for example, the plasma-based source of the invention provides one or more directed particle beams having a distribution of incident angles, such as a distribution of incident angles characterized between 0 and 90 degrees with respect to the sample surface normal.

In embodiments, the provided method further comprises directing one or more additional beams onto the substrate surface, wherein the addition beams are one or more particle beams, radiation beams or a combination thereof. In an embodiment, for example, the one or more additional beams are characterized by at least one beam property that differs from the one or more beam properties of the directed energetic particle beam. In an embodiment, the one or more additional beams are directed energetic particle beams. In embodiments, the one or more additional beams is a focused ion beam, a broad ion beam, a thermal beam, a plasma generated beam, an optical beam or any combination of these.

Selection of process parameters for some embodiments is made on the basis of the composition of the substrate and the type and properties of the surface structures to be generated. Table 1 provides a summary of process parameter ranges useful for certain application of the present methods and systems.

TABLE 1
Process parameter ranges for substrate classes

| | | | | | |
|---|---|---|---|---|---|
| | | | Nanorods/ | | |
| Structure | Nanoripples | Nanowalls | Nanocones | Nanoplates | Others |

1a. Substrate: TITANIUM

| | | | | | |
|---|---|---|---|---|---|
| Fluence | 10¹⁶ to 10²⁰ | 10¹⁶ to 10²⁰ | 10¹⁶ to 10²⁰ | 10¹⁶ to 10²⁰ | 10¹⁶ to 10²⁰ |
| (cm⁻²) | | | | | |
| Energy | 0.05 to 1.0 | 0.05 to 1.0 | 0.05 to 1.0 | 0.05 to 1.0 | 0.05 to 1.0 |
| (keV) | | | | | |
| Incident Angle | 65 to 80° | 0 to 45° | 0 to 45° | 0 to 45° | 0 to 80° |
| Flux | 10¹¹ to 10¹⁸ | 10¹¹ to 10¹⁸ | 10¹¹ to 10¹⁸ | 10¹¹ to 10¹⁸ | 10¹¹ to 10¹⁸ |
| (cm⁻²s⁻¹) | | | | | |
| Time | 1 to 10⁴ | 1 to 10⁴ | 1 to 10⁴ | 1 to 10⁴ | 1 to 10⁴ |
| (s) | | | | | |
| Ratio of ions | 0.01-0.9 | 0.01-0.9 | 0.01-0.9 | 0.01-0.9 | 0.01-0.9 |
| to neutrals | | | | | |
| Extraction | 10 to 1000 | 10 to 1000 | 10 to 1000 | 10 to 1000 | 10 to 1000 |
| Voltage | | | | | |
| (V) | | | | | |

1b. Substrate: MAGNESIUM and MAGNESIUM OXIDE

| | | | | | |
|---|---|---|---|---|---|
| Fluence | 10¹⁶ to 10²⁰ | 10¹⁶ to 10²⁰ | 10¹⁶ to 10²⁰ | 10¹⁶ to 10²⁰ | 10¹⁶ to 10²⁰ |
| (cm⁻²) | | | | | |
| Energy | 0.1 to 2.0 | 0.1 to 2.0 | 0.1 to 2.0 | 0.1 to 2.0 | 0.1 to 2.0 |
| (keV) | | | | | |
| Incident Angle | 45 to 80° | | | | |
| Flux | 10¹¹ to 10¹⁸ | 10¹¹ to 10¹⁸ | 10¹¹ to 10¹⁸ | 10¹¹ to 10¹⁸ | 10¹¹ to 10¹⁸ |
| (cm⁻²s⁻¹) | | | | | |
| Time | 1 to 1800 | 1 to 1800 | 1 to 1800 | 1 to 1800 | 1 to 1800 |
| (s) | | | | | |
| Ratio of ions | 0.01-1.0 | 0.01-1.0 | 0.01-1.0 | 0.01-1.0 | 0.01-1.0 |
| to neutrals | | | | | |
| Extraction | 10 to 1000 | 10 to 1000 | 10 to 1000 | 10 to 1000 | 10 to 1000 |
| Voltage | | | | | |
| (V) | | | | | |

Methods of the invention may be accomplished by a number of different tools. In FIG. 1, at least one embodiment of a system for carrying out DPNS and/or DSPNS on a substrate (product) is shown. The system, which may also be referred to herein as “Plasma3D), described below in connection with FIG. 1, includes a design that allows for treating the outer surface of a substrate by generating a plasma from a low temperature plasma source in a cylindrical geometry, and draws the particles inward by applying voltages to the grid, creating a “beam” from all directions that is capable of interacting with the outer surface of the substrate. It will be appreciated that the systems of the invention are particularly adapted to non-planar substrates and substrate surfaces. It will be further appreciated that although FIG. 1 shows a cylindrical design, other shapes for systems of the present invention for non-planar substrates and surfaces are envisioned including annuli, polygonal prisms, cones or spheres.

FIG. 1 shows a schematic of the components used to carry out an exemplary process according to the invention, e.g. an inward-facing plasma source to treat an outer surface of a substrate. As shown in FIG. 1, at least one embodiment of a system 100 for carrying out DPNS and/or DSPNS on product (substrate) 190 includes a housing 110 in a cylindrical geometry. Housing 110 includes an outer housing wall 120, gas inlet 130, waveguide 140, and inner housing wall 150. Housing 110 generates plasma 160 in a cylindrical geometry, using e.g. microsaves 106. The particles in plasma 160 generated from the gas in the gas inlet 130 is drawn inward as ion beams 180 from all directions onto product (substrate) 190 by electrified grid(s) 170 which then modify the surface to create nanoscale features on surface 190 in accordance with the invention.

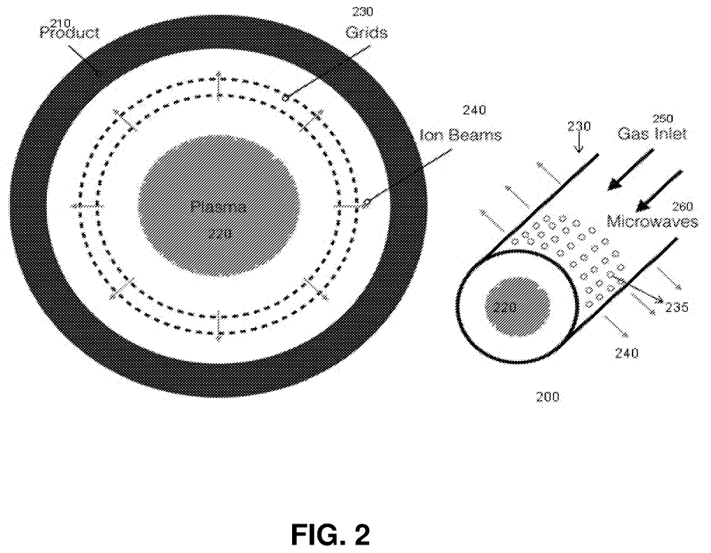

FIG. 2 shows an alternative design for substrates (products) having an inner surface to be treated. Exemplary products (substrates) include medical devices with an inner surface such as stents. It will be appreciated that although FIG. 1 shows a cylindrical design, other shapes are envisioned including annuli, polygonal prisms, cones or spheres.

FIG. 2 shows the schematic of outward-facing plasma source to treat an inner surface of a substrate (product) 210. In FIG. 2, plasma 220 is generated within a set of cylindrical grids 230 in the which then direct ion beams 240 onto the inner surface of product (substrate) 210. Voltages can then be applied to grid(s) 230 to extract particles in all outward directions. Inserting this plasma source inside of substrate (product) 210 allows the inner surface of substrate 210 to create nanoscale features on surface of substrate 210 in accordance with the invention. Inset to FIG. 2 shows an expanded view of plasma source 200 to insert into product (substrate) 210 having cylindrical grid(s) 230 with a plurality of openings 235 surrounding a plasma 220 having inlets 250 for gas and 260 for microwave to allow ion beams 240 directed onto the inner surface of product 210.

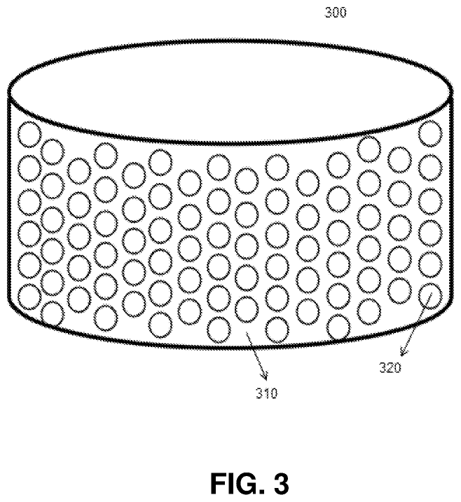

For both of the above designs, cylindrical conductive grids are used to manipulate various species within the plasma. FIG. 3 shows another representation of cylindrical conductive grids. Cylindrical conductive grid 300 has a body 310 containing a plurality of openings 320.

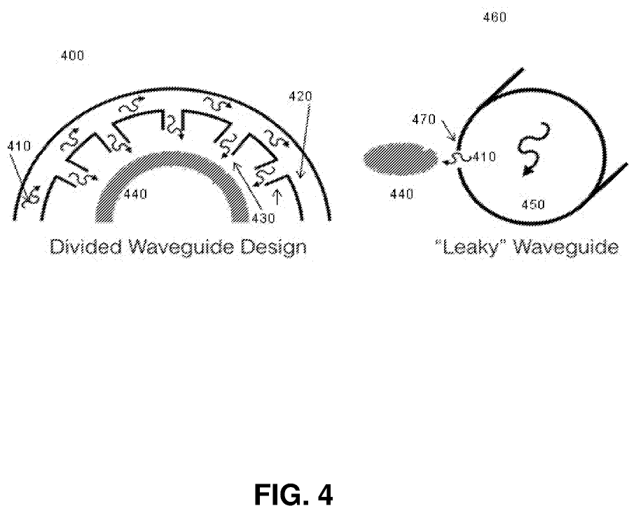

In one embodiment, a method for generating a directed particle beam using a low temperature plasma source useful for methods described herein and in design shown herein to accomplish the methods of the invention is a microwave source that excites the gas leading to plasma formation. Other methods known in the art for generating a low temperature plasma source are also consistent with the methods of the invention. To achieve the desired properties of the plasma, it is desirable to provide a waveguide that will distribute the microwaves evenly in the appropriate and desired geometry. A number of embodiments of designs for waveguides according to the invention are provided herein. In one embodiment, see FIG. 4 left side, the design for the divided cylindrical waveguide 400 includes a primary path 420 around the circumference of cylindrical waveguide 400, with multiple divisions 430 to direct the microwaves 410 into the gas and create plasma 440. In another embodiment, see FIG. 4 right side, of a “leaky” cylindrical waveguide 460 includes primary path 450, having with a constant small opening 470 along the inner circumference of cylindrical waveguide 460 to evenly distribute the microwaves 410 to create plasma 440. These are shown on the left and right sides, respectively, in FIG. 4.

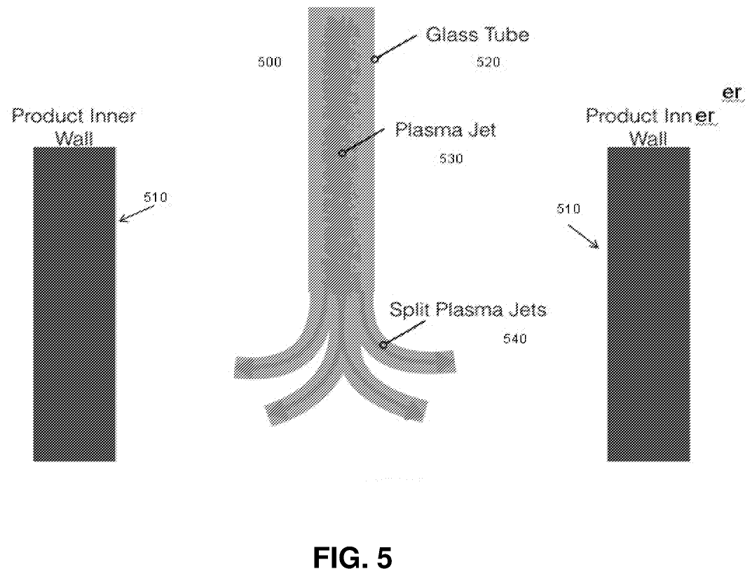

FIG. 5 shows a schematic of the components used to carry out another exemplary process according to the invention, e.g. creating an inward-facing plasma source in the form of plasma jets to treat an inner surface of a substrate. As shown in FIG. 5 provided is a schematic for a microplasma source 500 that operates at atmospheric pressure. In microplasma source 500, a jet of plasma 530 is generated in a glass tube 520 that splits the jet 530 into a plurality of directions, as desired for allowing for the processing of an inner surface 610 of a substrate (e.g., product).

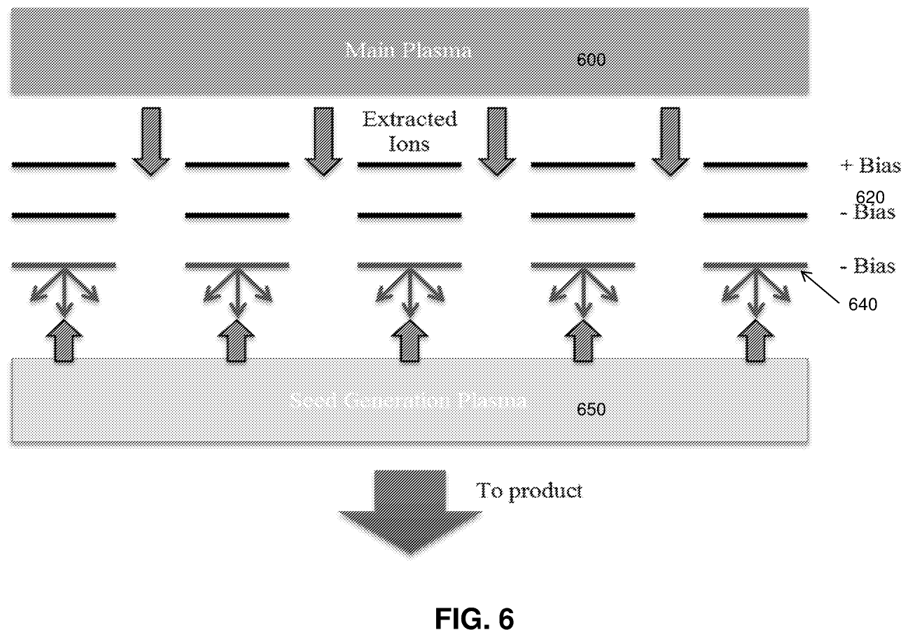

In another embodiment for accomplishing the methods of the invention, called herein “Plasma SEED”, includes the addition of components to add or “seed” ultra-low energy, chemically reactive surfactants to the plasma as it interacts with material surfaces. See FIG. 6. The ultra-low energy surfactants allow for the ability to modify surfaces directly through chemical and surface free energy interactions, adding to the tunability (or capabilities) of DPNS. The combination of particle-beam “states” are designed deliberately and dictated by the energy density deposition known to drive specific metastable irradiation-driven phases that drive variation in composition, surface topology/morphology/topography and surface charge density among other surface states. This can exploit the particle-surface state conditions that drive bottom-up atom-by-atom self-organization by a balance of energy deposition and mass redistribution resembling larger physical-scale technologies such as additive manufacturing. Recipes can be designed and controlled ultimately by computer design to guide 3D structure fabrication from a few nanometers to microns with a broad application sector including: energy, biomaterials, nanophotonics, nanoelectronics and self-healing materials, among others.

Metal seeds can be introduced in the plasma by exploiting electrode extraction operation at lower operating voltages. In dual-grid systems, the plasma generation to generate plasma 600 can occur just outside the grid assemblies 620, causing the ions to be accelerated into the outer most grid 640 resulting in erosion of the grid material 640 towards the substrate (product) via a seed generation plasma 650 In the Plasma3D configuration the third grid is biased negatively and is designed to operate as the “seeding” grid.

The addition of a second waveguide is provided to allow controlled plasma generation in-between the seeding grid and product, with the seeding grid 640 having a variable negative voltage in order to attract ions for sputtering of the grid 640. This would introduce a flux of electrons to the product surface, but should not have significant effects. The third grid 640 is required to allow for the controlled sputtering of the seed material onto the substrate (product) surface (increase the negative bias on the third grid in order to increase the sputter yield/seed ion flux). The grid 640 is designed to have a given amount of seed material in form of a coating on the substrate (product)-facing side.

In a separate configuration, Plasma SEED, will consist of a source of evaporated sputtered material from one to four points along the circumference of the generated plasma where the “seed” particles are coupled to the non-equilibrium plasma and thus driven by the hot electrons to the plasma sheath and eventually to the surface. The three-grid system is still used in this configuration to control both the flux and energy of the particles. In addition, the third grid is designed with a material that enables neutralization of a fraction of the ion flux from the plasma. This grid acts to control the ion/neutral ratios that eventually reach the product material surface.

The substrates to treat according to the invention include a broad range of devices including medical/pharmaceutical applications. A few examples of specific devices include; biosensors, catheters, stents, and even artificial organs.

The invention may be further understood by reference to the following nonlimiting Examples that expand on certain aspects and embodiments of the invention.

### EXAMPLES

The wide scope and variety of surface structures can be synthesized by DPNS, depending on desired function or exemplary embodiments of structure and function. For example, to elicit an immune-modulated response for macrophage phenotype that have anti-inflammatory osseoconductive properties, an exemplary embodiment of these structures would be made of medical-grade Ti alloy exposed to a particular fluence, angle of incidence, energy and species in the energetic particle beams from DPNS methods. The methods can be broadly used to create structures of a wide range of material classes and surfaces.

### Example 1.1 Parameter: Gas (Kr)

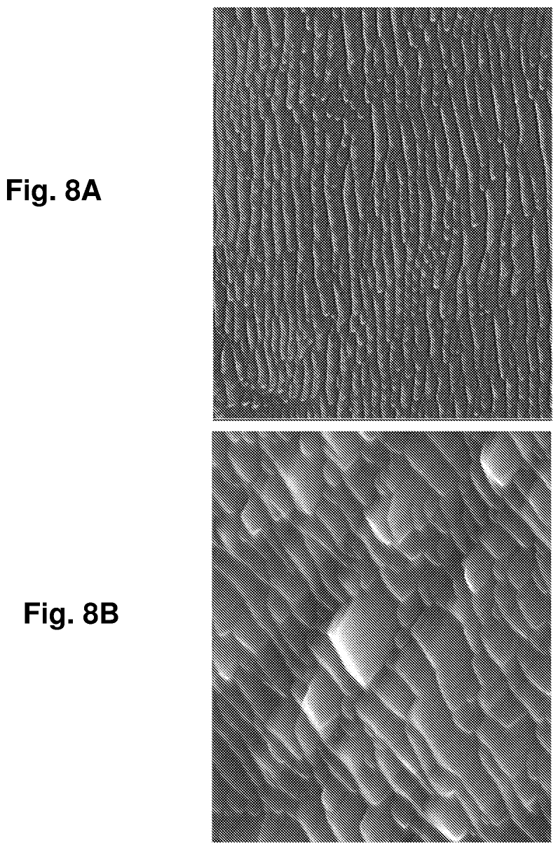

a) Parallel Nano-walls (Kr): an array of (parallel) wall like structures having a height of 3 to 25 nm, with a length ranging from 10 to 40 nm, said ridge composed primarily of titanium alloy (FIGS. 8a and 8b) were created. Such structures are formed using a method comprising of Kr ions, incident at an angle with respect to surface normal=60°, Energy: 1 keV, Fluence: 1×1018 cgs with an effective surface area of 2-5×1016 nm2. FIG. 8A shows long nano-walls and FIG. 8B shows short nano-walls (MAG ×80,000).

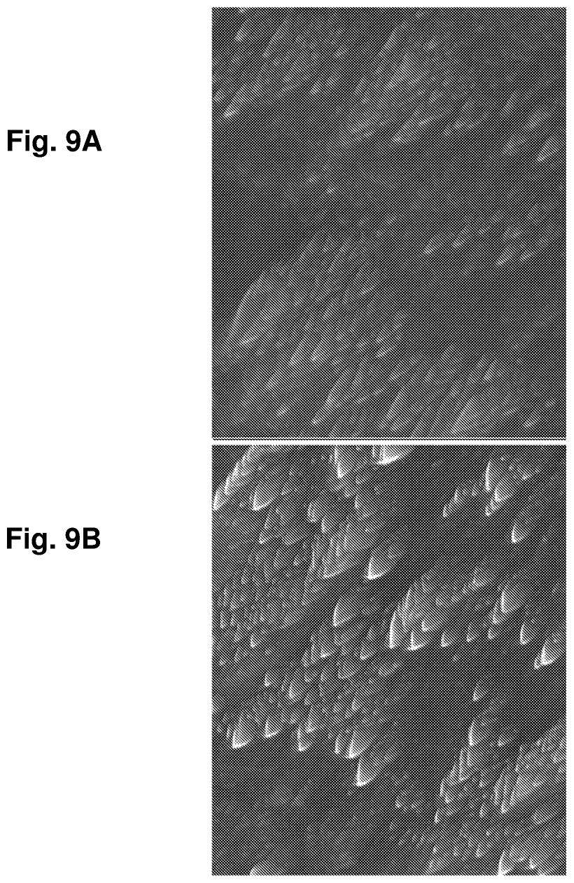

b) Nano-cones (Kr): Structures which resemble sharp-like cones. These pointed sharp regions are inclined towards the incident beam direction (FIG. 9A and FIG. 9B). The dimension of these cones are in the range of 2 to 100 nm, with a length ranging from 10 to 40 nm. These cones consist of Ti alloy. Such structures are formed using following parameters, Gas: Kr, Angle: 60°, Energy: 1 keV, Fluence: 1×1018 cgs. FIG. 9A shows narrow nano-cones and FIG. 9B shows wide nano-cones (MAG ×50,000).

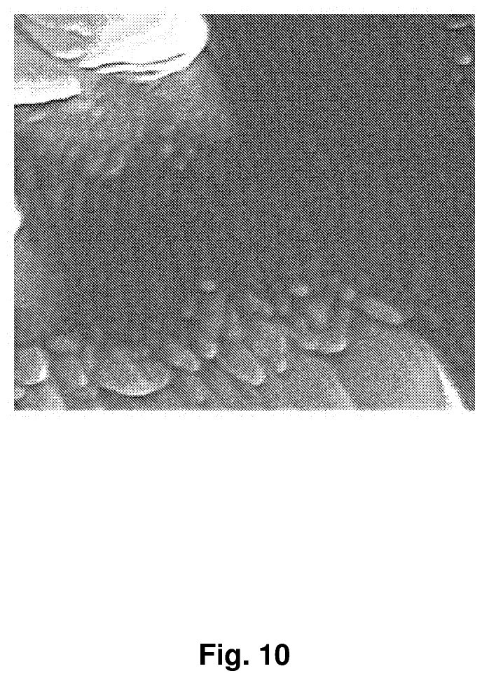

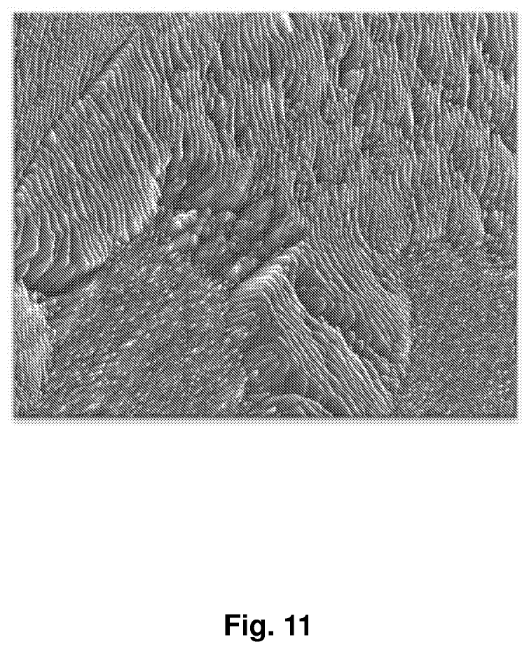

c) Nano-ripples (Kr): In the midst of nano-pillar and nano-cones, we also observe nanoripples as shown in FIG. 10 (MAG ×120,000). Such structures are formed using following parameters, Gas: Kr, Angle: 60°, Energy: 1 keV, Fluence: 1×1018 cgs.

### Example 1.2 Parameter: Gas (Ar)

a) Nano-walls (Ar): An array of (parallel) wall like structures having a height of 3 to 100 nm preferably, with a length ranging from 10 to 40 nm, said ridge composed primarily of titanium alloy (FIG. 11, MAG ×30,000). Such structures are formed using following DIS parameters—Gas: Ar, Angle: 60°, Energy: 1 keV, Fluence: 1×1018 cgs.

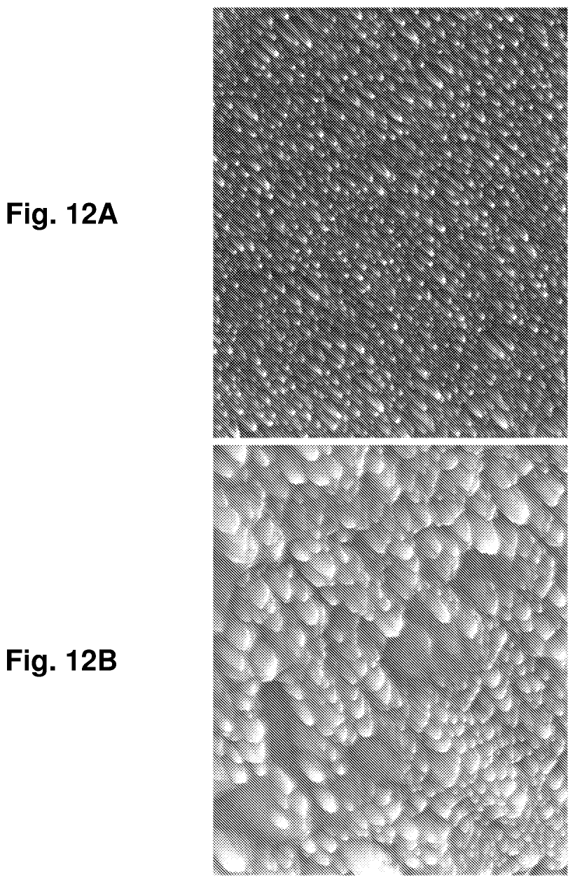

b) Nano-cones (Ar): Structures which resemble sharp-like cones. These pointed sharp regions are inclined towards the incident beam direction (FIG. 12A shows narrow nano-cones and FIG. 12B; shows wide nano-cones (resol: 500 nm)). The dimension of these cones are in the range of 2 to 100 nm, with a length ranging from 10 to 40 nm. These cones consist of Ti alloy. Such structures are formed using following parameters—Gas: Ar, Angle: 60°, Energy: 1 keV, Fluence: 1×1018 cgs.

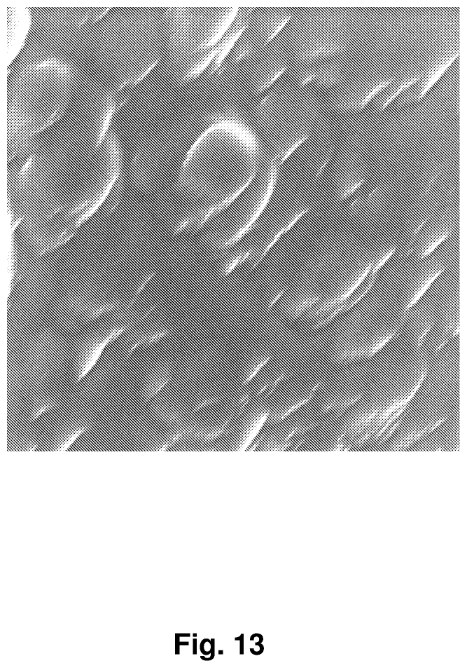

c) Round-plate formation (Ar): The structures resemble round plate formation (FIG. 13, 1 micron resolution). The diameter is around 50 nm. Such structures are formed using following parameters, Gas: Ar, Angle: 60°, Energy: 1 keV, Fluence: 1×1018 cgs.

### Example 1.3 Parameter: Fluence 7.5×1017 cgs (Ar)

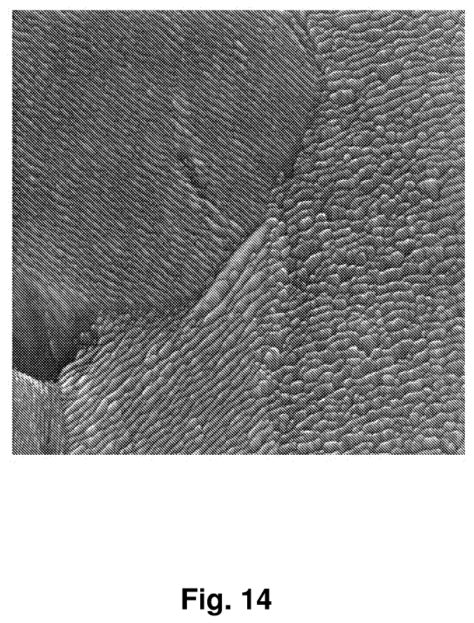

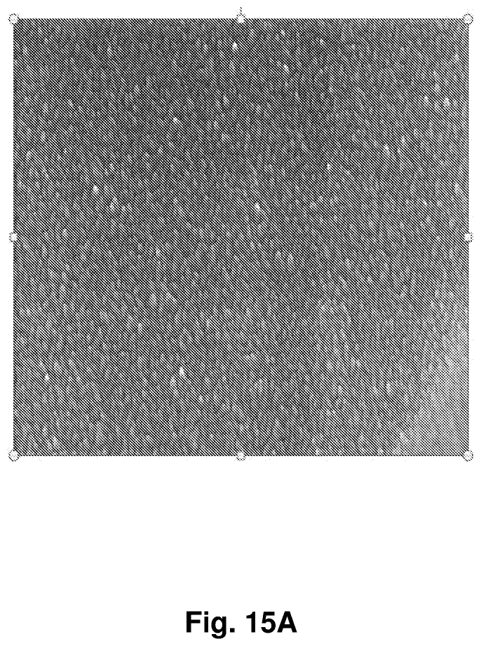

a) Nano-walls (Ar): An array of (parallel) long and short wall like structures having a height of 3 to 100 nm preferably, with a length ranging from 10 to 40 nm, said ridge composed primarily of titanium alloy (FIG. 14 showing nanowalls resol 1 micron). Such structures are formed using following DIS parameters—Gas: Ar, Angle: 60°, Energy: 1 keV, Fluence: 7.5×1017 cgs. The walls are oriented in different directions and sizes.

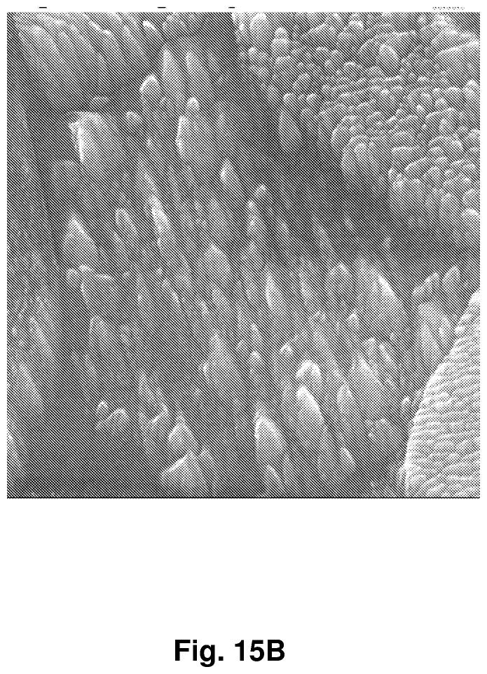

b) Nano-cones (Ar): Structures consist of very narrow and wide cones (FIG. 15A, showing narrow cones, and FIG. 15B, showing wide cones, (resol: 500 nm)). These cones consist of Ti alloy. Such structures are formed using following DIS parameters—Gas: Ar, Angle: 60°, Energy: 1 keV, Fluence: 7.5×1017 cgs.

### Example 1.4 Parameter: Fluence 5×1017 cgs (Ar)

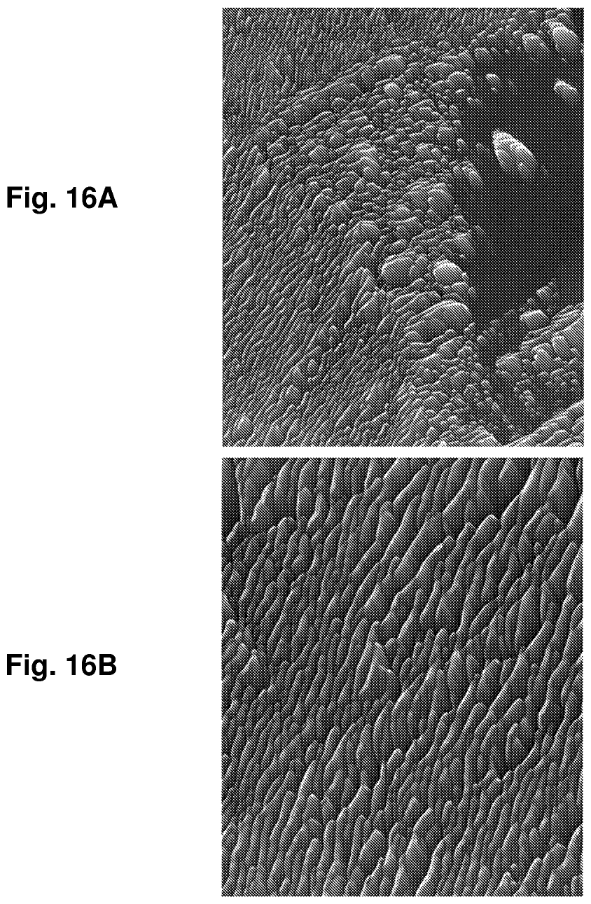

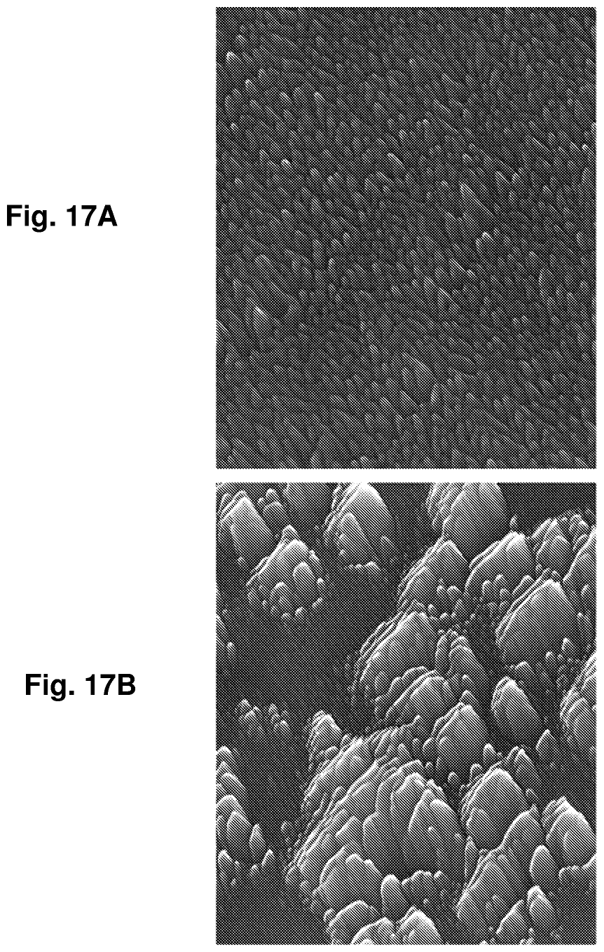

a) Nano-walls (Ar): An array of (parallel) long and short wall like structures having a height of 3 to 100 nm preferably, with a length ranging from 10 to 40 nm, said ridge composed primarily of titanium alloy (FIG. 16A, long nanowalls, and FIG. 16B, short nanowalls, resolution 500 nm). Such structures are formed using following DIS parameters—Gas: Ar, Angle: 60°, Energy: 1 keV, Fluence: 5×1017 cgs. The walls are oriented in different directions and sizes.

### Example 1.5 Parameter: Fluence 2.5×1017 cgs (Ar)

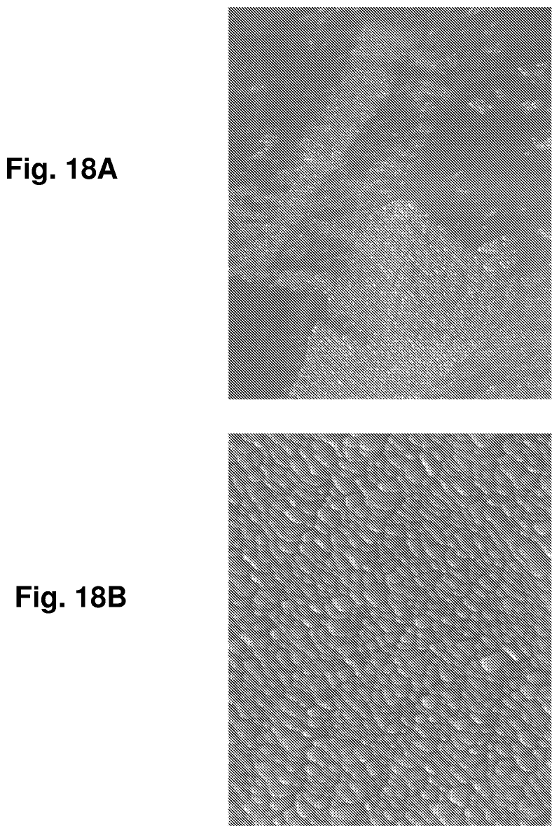

a) Nano-walls (Ar): The surface consists of smooth and nanostructured surface composed primarily of titanium alloy (FIG. 18A: small nano-walls with smooth surface (resol: 2 microns) and FIG. 18B: small nano-walls at high resolution (resol: 500 nm)). Such structures are formed using following DIS parameters—Gas: Ar, Angle: 60°, Energy: 1 keV, Fluence: 2.5×1017 cgs. The walls are oriented in different directions and sizes

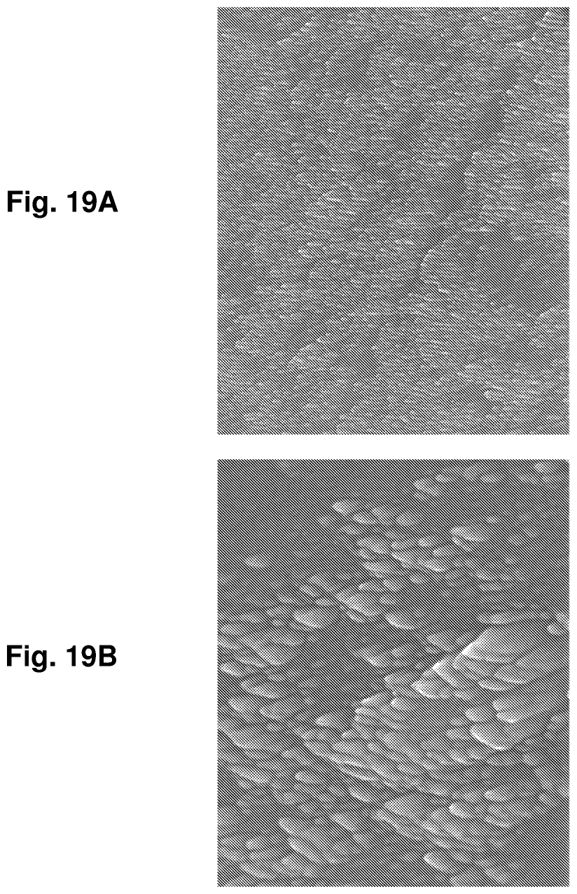

b) Nano-cones (Ar): The surface consists of smooth and nanostructured surface. Structures consist of very narrow and wide cones (FIG. 19A: narrow nano-cones and FIG. 19B: wide nano-cones (resol: 500 nm)). These cones consist of Ti alloy. Such structures are formed using following DIS parameters—Gas: Ar, Angle: 60°, Energy: 1 keV, Fluence: 5×1017 cgs.

### Example 1.6 Parameter: Fluence 1×1017 cgs (Ar)

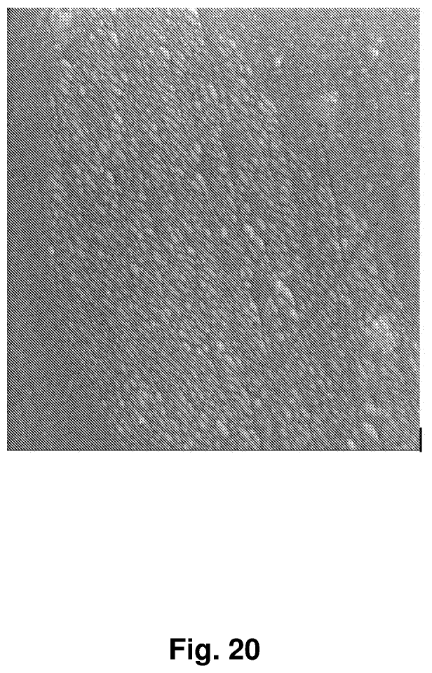

a) Nano-walls (Ar): The surface consists of fine nanostructures which are seen at specific regions on the surface. Due to their fine nature we cannot measure their dimensions. These structures are composed primarily of titanium alloy (FIG. 20 showing nano-wall formation at high resolution (resol: 500 nm)). Such structures are formed using following DIS parameters—Gas: Ar, Angle: 60°, Energy: 1 keV, Fluence: 1×1017 cgs.

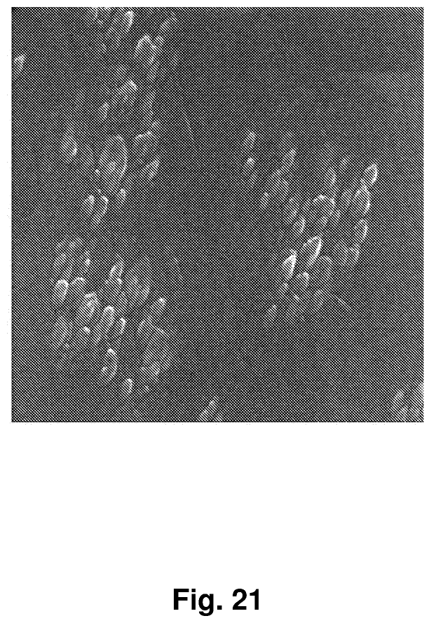

b) Nano-cones (Ar): Structures consist of cones which are non-uniform on the surface (FIG. 21, showing nano-cone formation at high resol (resol: 500 nm)). These cones consist of Ti alloy. Such structures are formed using following DIS parameters—Gas: Ar, Angle: 60°, Energy: 1 keV, Fluence: 1×1017 cgs.

### Example 1.7 Angles 0° to 80° (Ar)

TABLE 1
Irradiation Parameters (1-keV Ar+) on Ti6Al4V Samples

| | Energy | Flux (ions | Fluence | Incidence | Time |
|---|---|---|---|---|---|
| Samples | (keV) | sec cm ⁻²) | (cm⁻²) | angle(°) | (s) |
| S1 (Ti6) | 1.0 | 6.51E14 | 2.5E17 | 0° | 384.0 |
| S2 (Ti6) | 1.0 | 3.29E14 | 2.5E17 | 30° | 758.8 |
| S3 (Ti6) | 1.0 | 6.44E14 | 2.5E17 | 60° | 387.9 |
| S4 (Ti6) | 1.0 | 6.55E14 | 2.5E17 | 80° | 381.6 |

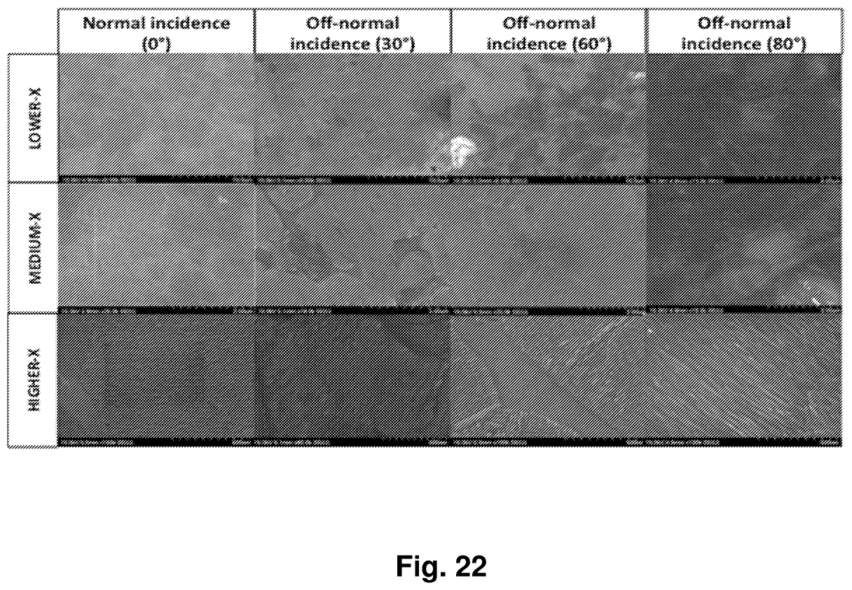

Surface structural modifications and nanostructuring due to DIS of Ti6Al4V are summarized in FIG. 22. At normal incidence an organized dot-like structure formed alongside “broken” nanoscale ripples on the original grains is observed. Comparison with the original surface microstructure, the self-organized dot-like structures appear concentrated in the a phase (grains and Widmanstätten plates), whilst the more irregular damage seems to be preferentially located within β phase matrix. Furthermore, the nanostructures at normal incidence appear to have some preferential growth depending on grain orientation. As the incident angle is increased, the stabilization of surface ripples is observed. These incipient nano-ripples can be also explained due to the activation of the terrace diffusion barrier and of the Ehrlich-Schwoebel barrier which opens the possibility for the development of the surface instability connected to ripple formation. At normal incidence, the features produced by ion sputtering reflect the surface symmetry and are aligned along energy preferred crystallographic orientation and enhanced surface recombination, which can lead to partial nano-dot and nano-ripple formation or in some cases complete smoothening of the surface.

In FIG. 22 the nano-structure features observed produced by an increase of the ion-beam incident angle to 60° are very similar to those observed for 30°. Due to the increased incident angle, it appears that curved ripples are even more important than in the case of the 30° incident ion-beam angle. Again, this mixed pattern formation indicates the competition between those above-mentioned mechanisms: a diffusive regime along preferential crystallographic directions and erosive regime with nanostructure's wave vector aligned parallel the incident ion-beam. The Ti β (bcc) phase still appears insensitive to any nano-structuring due to ion irradiation at the 60° incidence.

At the highest incident angle (e.g. 80° incidence), a new trend in the resulting nano-pattern is obtained (see FIG. 22). Namely the α-phase of Ti6Al4V alloy was not the only phase material constituent responding to Ar+ ion irradiation, but also the Ti β (bcc) phase. At this grazing incidence new nanoscale ripples are observed in both phases. The ripples in both phases are found to have their wave vector aligned with the ion-beam direction. The transition angle between perpendicular and parallel wave-vector ripple formation with respect to the incident ion-beam direction is therefore found to be between 60° and 80° incidence. This transition is also indicative of an observed transition for Ti-based alloys between surface and sub-surface diffusive mechanisms to erosion-dominated mechanisms dominant at grazing incidence. Furthermore, at grazing incidence the separation distance between ripples decreases resulting in well-aligned ripple structures that are about 50-nm thin and with lengths close to 0.1-0.5 μm. Another important result here is that the dominating erosive processes found during grazing incidence irradiation can be achieved at room temperature. These conditions inhibit thermally-activated diffusion processes, which tend to smooth the surface and to orient the nanostructures along the preferential thermodynamic orientations. This specific response indicates that, under erosive sputtering conditions, it is possible to grow nanostructures, which can be aligned along thermodynamically unfavoured directions.

FIG. 22 shows SEM images showing the evolution of surface nano-patterning of Ti6Al4V samples for different incidence angles with Ar+ irradiation.

### Example 2. Crystallographic Structures

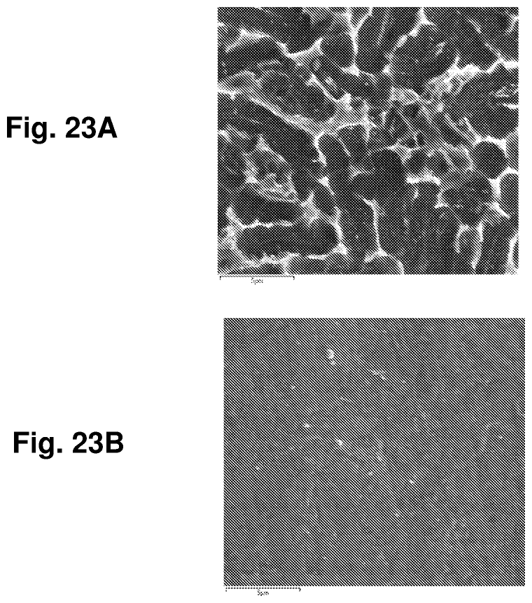

Considering the microstructure of unirradiated Ti6Al4V samples after proper etching procedures, one can observe a conventional α-β mill-annealed alloy (see FIG. 23A-23B) consisting of a phase (hcp), equiaxial grains and Widmanstäten plates, dispersed in an untransformed β matrix (bcc). FIG. 23A-23B show surface characteristics of Ti6Al4V samples before DIS processing: 23A shows surface prepared up to mirror finishing; 23B shows biphasic (α (hcp) is grey and β (bcc) is white) structure of the alloy revealed after polishing and metallographic acid etching. This microstructure is the consequence of both heating and milling at the α-β thermodynamically stable region, and further slow cooling, allowing β-α transformation. This combination of phases and constituents results in an excellent balance between mechanical strength, toughness, ductility and fatigue resistance. Surface structural modifications and nanostructuring due to DIS of Ti6Al4V are summarized in FIG. 22.

Intrinsic to the DIS modification is its ability to only modify the first few 100's of nm and therefore not affect the optimized mechanical properties mentioned described above. The first observation relates to the effect normal incidence has on the modified surface. At normal incidence an organized dot-like structure formed alongside “broken” nanoscale ripples on the original grains is observed. Comparison with the original surface microstructure, the self-organized dot-like structures appear concentrated in the a phase (grains and Widmanstätten plates), whilst the more irregular damage seems to be preferentially located within β phase matrix. Furthermore, the nanostructures at normal incidence appear to have some preferential growth depending on grain orientation. However, normal incidence and low energy processes in some types of materials (e.g. Si) have resulted in the smoothening of the surface. Evidence for a resistance to patterning is found in the normal incidence case as well as more oblique angles for certain grains. This could be evidence of a balance between mass redistribution mechanisms that drive atoms on the surface to recombine with irradiation-driven surface vacancies leading to smooth surfaces. The fact that smooth surfaces only occur under certain grain orientations suggests that there is also a structure-driven relaxation mechanism coupled to the irradiation-driven mechanisms that lead to self-organized nanostructures. At normal incidence, the features produced by ion sputtering reflect the surface symmetry and are aligned along energy preferred crystallographic orientation and enhanced surface recombination, which can lead to partial nano-dot and nano-ripple formation or in some cases complete smoothening of the surface. Comparing to this work we find resistance to nanopatterning along specific grain orientation or microstructure phases.

Moreover, we discovered this morphological trend with incident ion-beam angle is dominant for the α-phase in the Ti6Al4V alloy meanwhile for the β (bcc) phase appears insensitive to this trend. The interaction and competition between the two mechanisms discussed above induces pattern formation observed in FIG. 22 and influences the resultant surface properties such as hydrophilicity and hydrophobicity.

In FIG. 22 are also depicted structures obtained from off-normal incidence starting with θ=30°. This change in the incident angle results in a predominant elongated rippled nanostructure at the irradiated surface mostly associated to the response of β(hcp) phase of the alloy. Given its polycrystallinity the observed nano-ripples were not perfectly perpendicular to the direction of irradiation. Most of them clearly appear with a shift of direction, which is a consequence of the coupling of incident energy deposition at the near surface along with surface diffusion being correlated to the movement of atoms associated with a particular crystallographic orientation. Interestingly, grains of the same β(hcp) phase were not transformed to nano-ripples, exhibiting a type of elongated nano-grains (short nano-rods). These nanostructures can be assumed as an intermediate nanostructure phase between curved “elongated” nano-ripples and long nano-rods in the same direction of the ion beam (see discussion later) consistent with an erosive-dominated and enhanced surface diffusion regime as described by the Bradley and Harper (BH) model. Another important point is the difference in response of metallic surfaces compared to semiconductor materials to ion irradiation. Metals has both higher diffusivity and the intrinsic non-directional nature of the metallic bond results in both resistance to amorphization and consequently a more sensitive dependence to crystallographic orientation. The formation of a pattern is thus dictated by: the surface curvature dependence of ion-induced sputtering and the presence of an extra energy barrier whenever diffusing atoms try to descend step edges. Formation of nano-ripples on Ti6Al4V due to off-normal ions incidence is consistent with those assumed mechanisms on metals: diffusive processes are mainly responsible for the formation of regular structures. However, here we have two new findings, which warrant brief discussion: those nano-rods observed in some grains of β(hcp) phase, and the clear insensitivity of β (bcc) phase in Ti6Al4V alloys to nano-structuring. These features (or the lack thereof) can be attributed to the influence of crystallographic orientation in conjunction with corresponding β(hcp) phase and β (bcc) phase.

### Example 3. Titanium Experimental Results

### Example 3.1 Parameter: Fluence 1×1018 cgs (Ar) 02

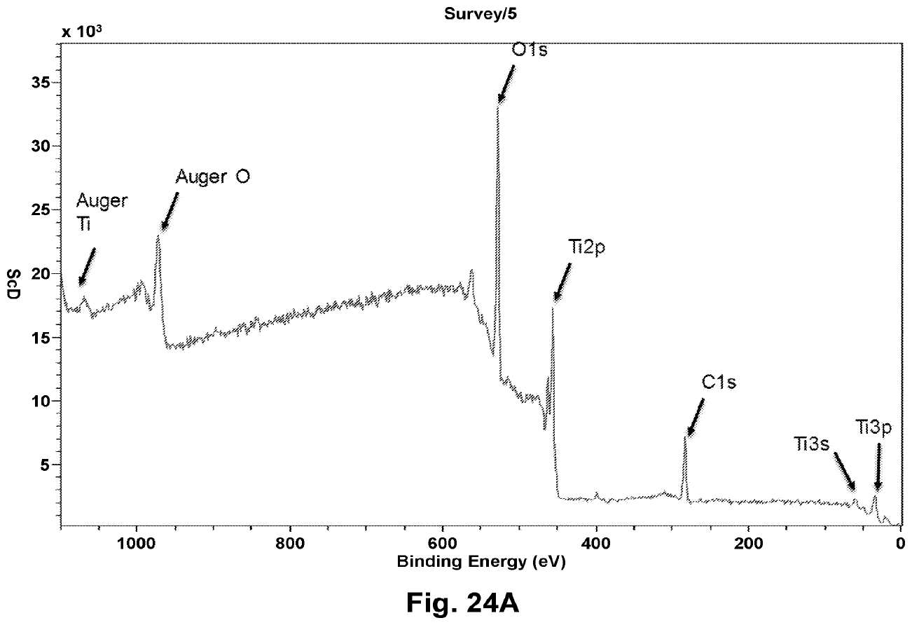

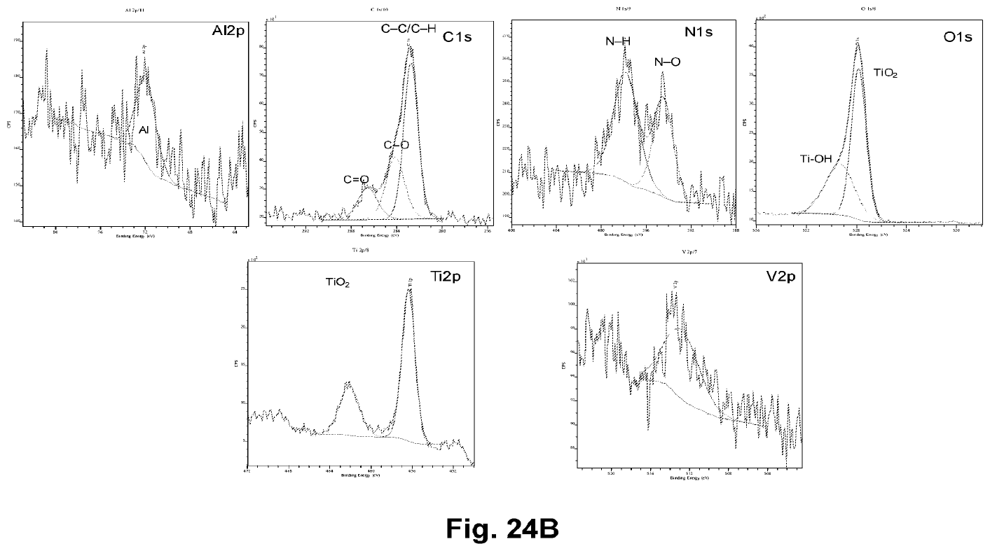

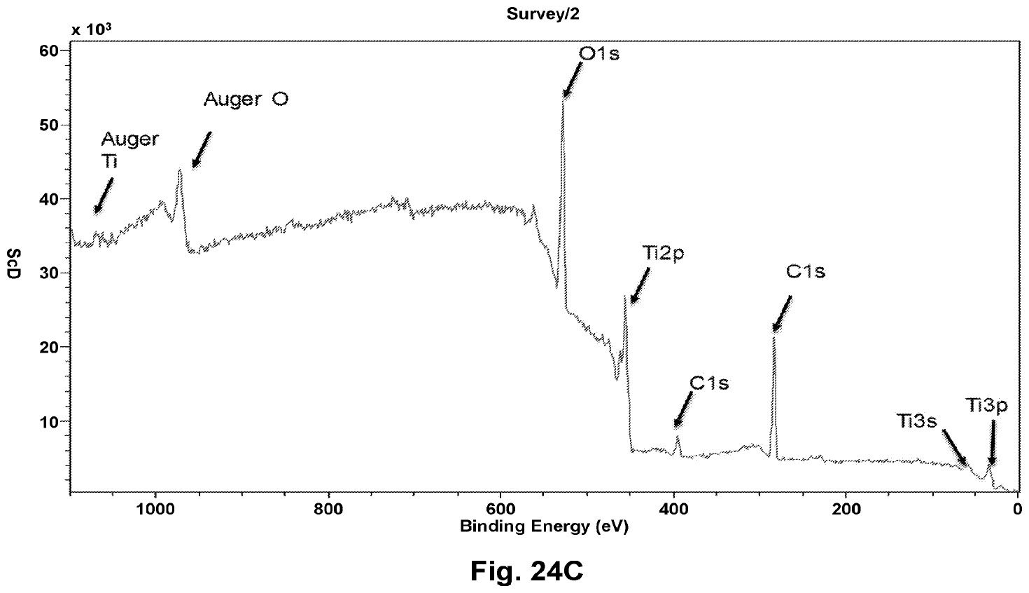

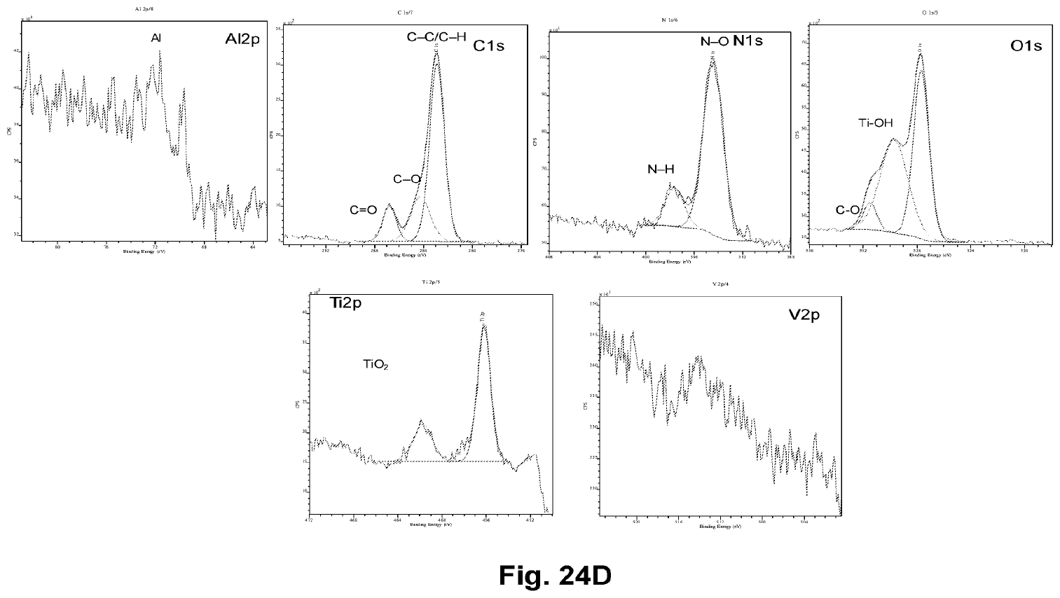

The survey scans for Ti alloy samples treated with a fluence of 1×1018 cgs for Ar irradiation at 60° is displayed in FIG. 24A. The results show the presence of Ti, C, O and N but shows the presence of no contaminants. The Al2p, C1s, N1s, O1s, Ti2p, and V2p are shown in FIG. 24B. The Al2p shows the presence of only Al after irradiation. There is no change in Ti2p and C1s compared to control. The 2 peaks of O1s are assigned to Ti—OH and TiO2. We now see a presence of V. After irradiation N1s shows the presence of N—H and N—O. The atomic concentration for control is summarized in Table 3.

TABLE 3
Atomic concentration for control Ti6Al4V

| Sample | O1s | C1s | Ti2p | N1s | V2p | Al2p |
|---|---|---|---|---|---|---|
| Ti—Ar-02 | 45.4 | 35 | 13.3 | 2.8 | 0.7 | 2 |

### Example 3.2 Parameter: Fluence 7.5×1017 cgs (Ar)_10

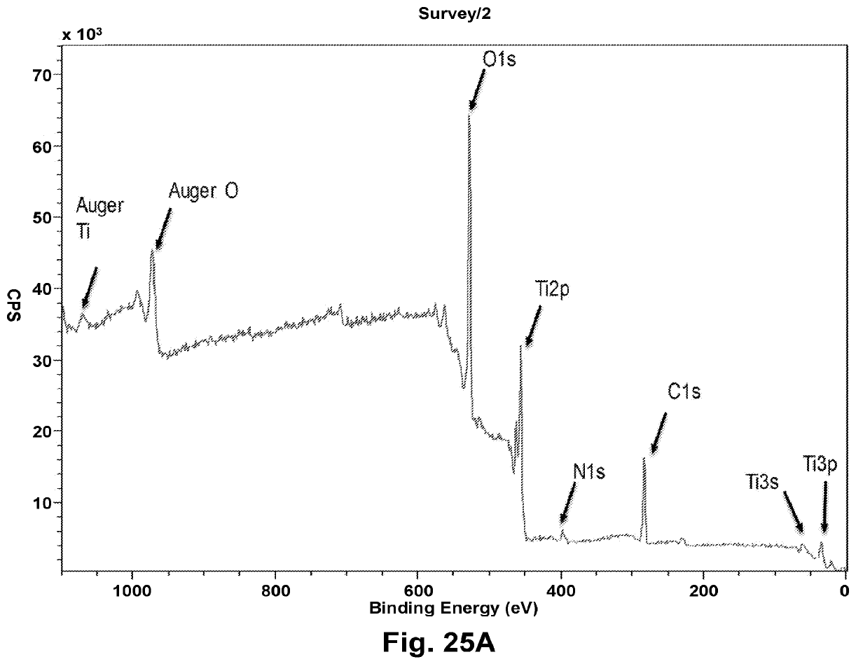

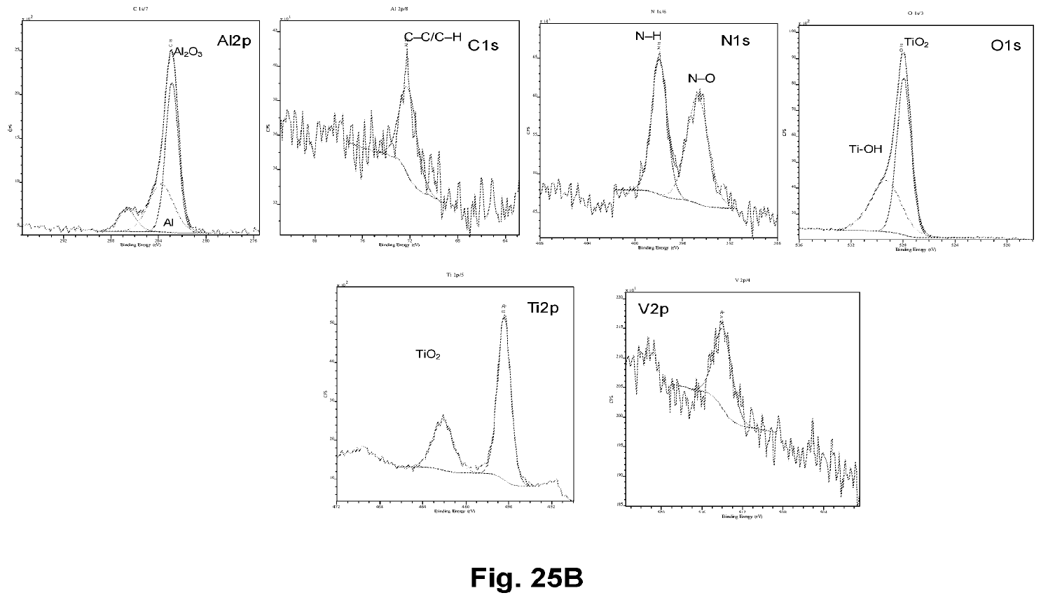

The survey scans for Ti alloy samples treated with a fluence of 7.5×1017 cgs for Ar irradiation at 60° is displayed in FIG. 25A. The results show the presence of Ti, C, O and. The Al2p, C1s, N1s, O1s, Ti2p, and V2p are shown in FIG. 25B. The Al2p shows the presence weak Al after irradiation. There is a minor decrease in C—O from C1s. There is no change in Ti2p. The O1 s shows the presence of one new peak which is assigned to C—O. We now see a weak presence of V. We observe a rise in N—O compared to N—H. The atomic concentration for control is summarized in Table 4.

TABLE 4
Atomic concentration

| Sample | O1s | C1s | Ti2p | N1s | V2p | Al2p |
|---|---|---|---|---|---|---|
| Ti—Ar-10 | 34 | 50.5 | 5.5 | 5.7 | 1 | 3.3 |

### Example 3.3 Parameter: Fluence 5×1017 cgs (Ar)_09

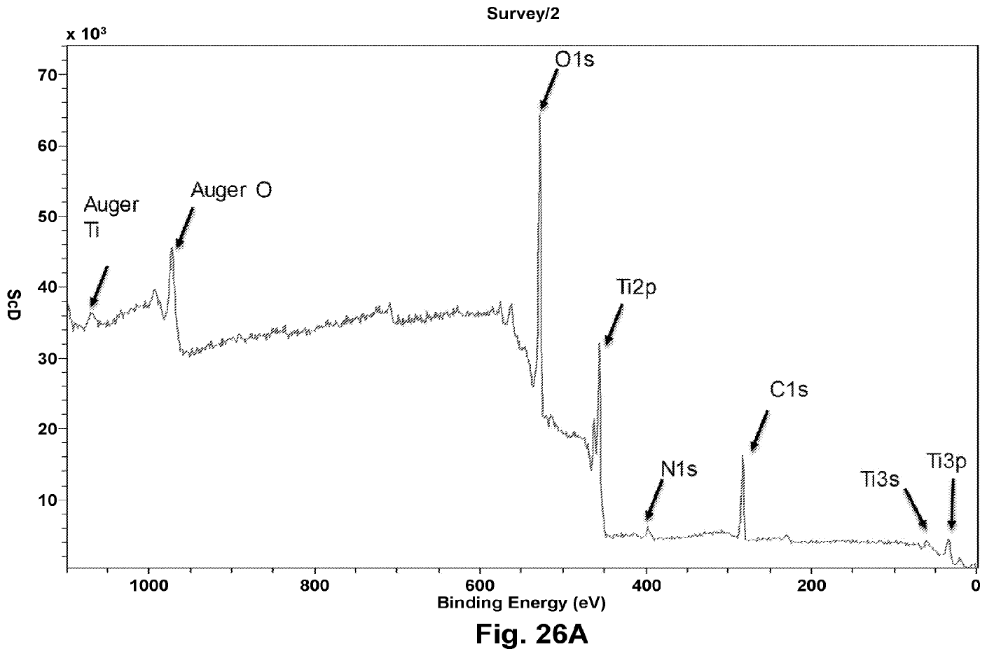

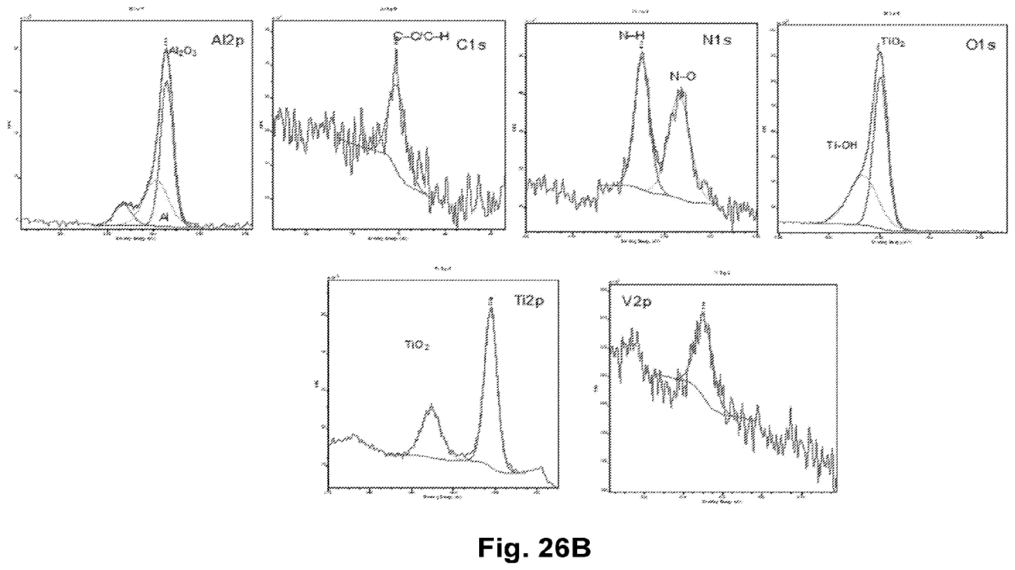

The survey scans for Ti alloy samples treated with a fluence of 5×1017 cgs for Ar irradiation at 60° is displayed in FIG. 26A. The results show the presence of Ti, C, O and N. The Al2p, C1s, N1s, O1s, Ti2p, and V2p are shown in FIG. 26B. The Al2p consist of three peaks, whereas C1s shows the presence of only one peak. There is no change in Ti2p. The O1s is composed of T—OH and Ti—O. We now see a weak presence of V. Meanwhile N1s is deconvoluted in to N—O and N—H. The atomic concentration for control is summarized in Table 5.

TABLE 5
Atomic concentration

| Sample | O1s | C1s | Ti2p | N1s | V2p | Al2p |
|---|---|---|---|---|---|---|
| Ti—Ar-07 | 41 | 44.1 | 11.28 | 1.6 | 0.24 | 1.6 |
| Ti—Ar-09 | 42.7 | 40 | 12.1 | 3.2 | 0.2 | 1.6 |

### Example 3.4 Parameter: Fluence 2.5×1017 cgs (Ar)_08

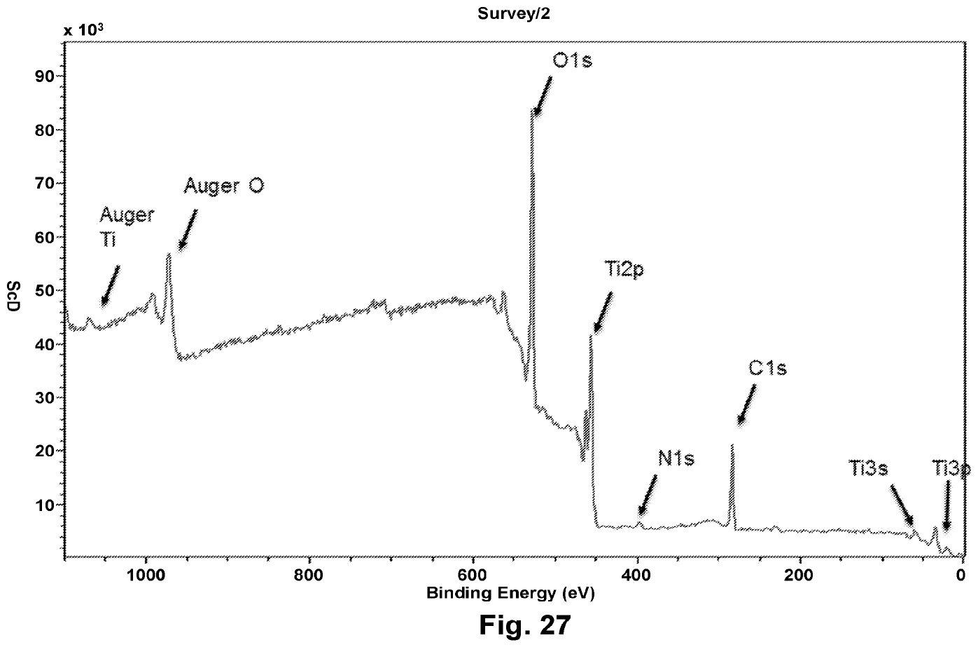

The survey scans for Ti alloy samples treated with a fluence of 2.5×1017 cgs for Ar irradiation at 60° is displayed in FIG. 27. The results show the presence of Ti, C, O and N. The Al2p, C1s, N1s, O1s, Ti2p, and V2p are not shown. The Al2p consist of only one Al peak. On the other hand, C1s is composed of three peaks. Meanwhile N1s is deconvoluted in to N—O and N—H. There is no change in Ti2p. The O1s is deconvoluted in to 3 peaks. We now see a weak presence of V. The atomic concentration for control is summarized in Table 6.

TABLE 6
Atomic concentration

| Sample | O1s | C1s | Ti2p | N1s | V2p | Al2p |
|---|---|---|---|---|---|---|
| Ti—Ar-08 | 43.2 | 39.1 | 12.6 | 2.2 | 0.5 | 2.07 |

### Example 3.5 Parameter: Fluence 1×1017 cgs (Ar)_07

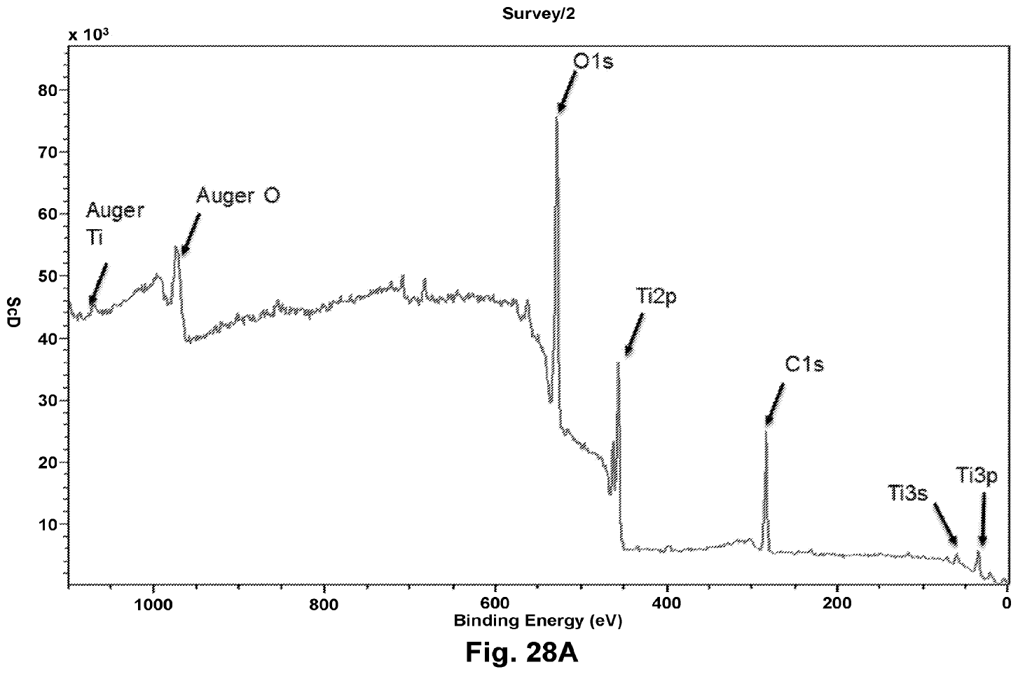

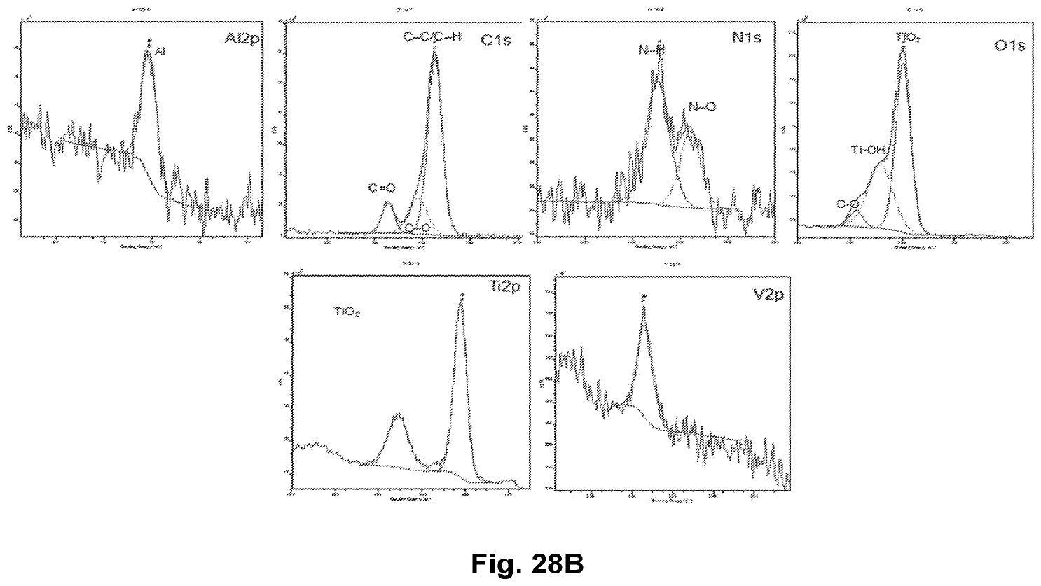

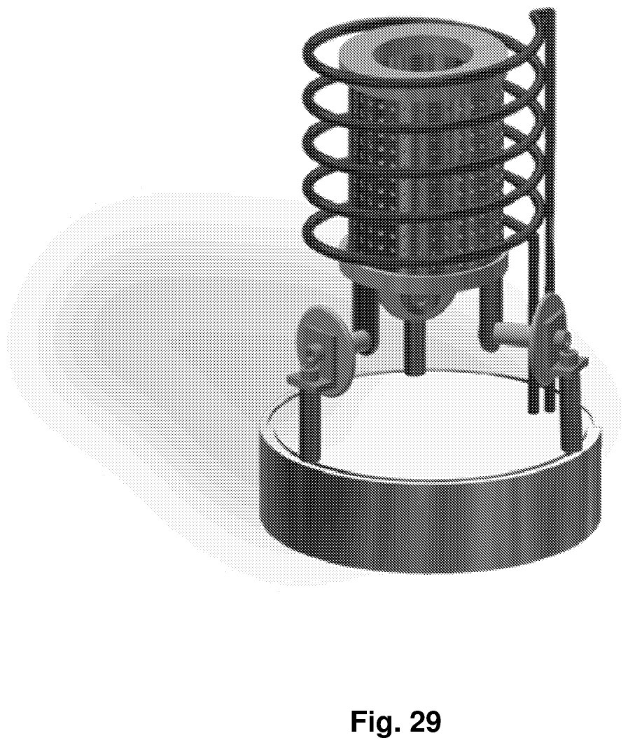

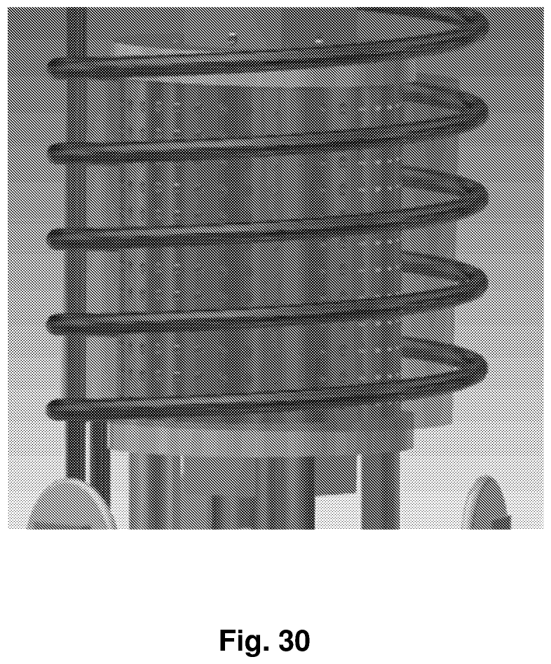

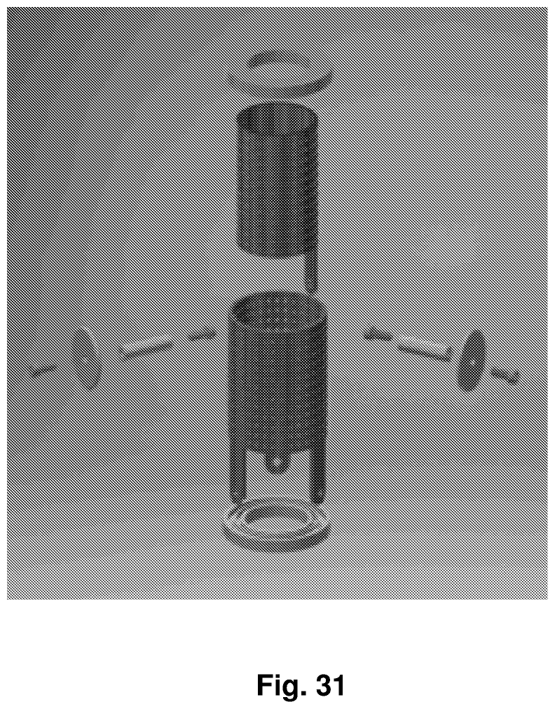

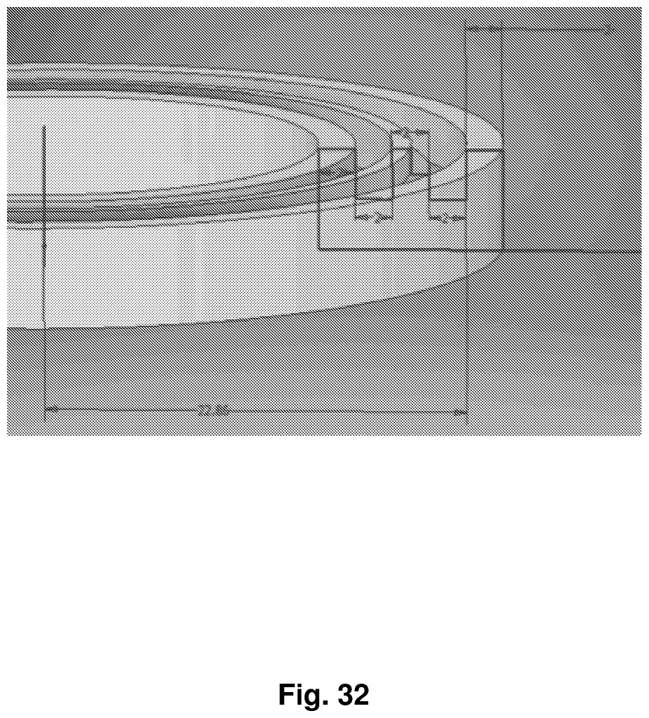

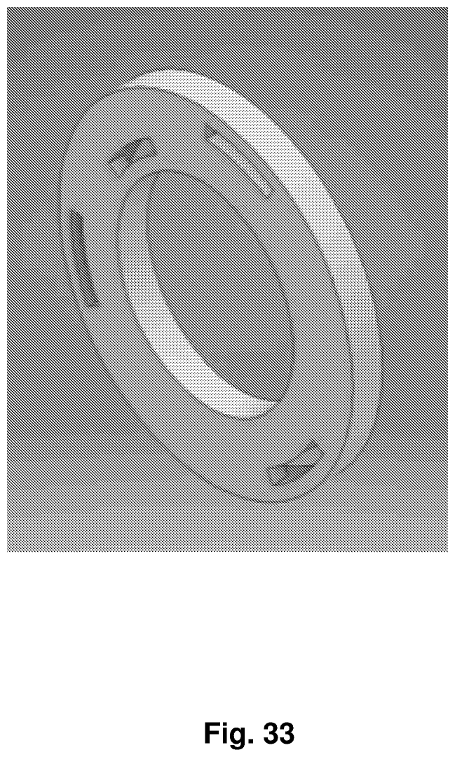

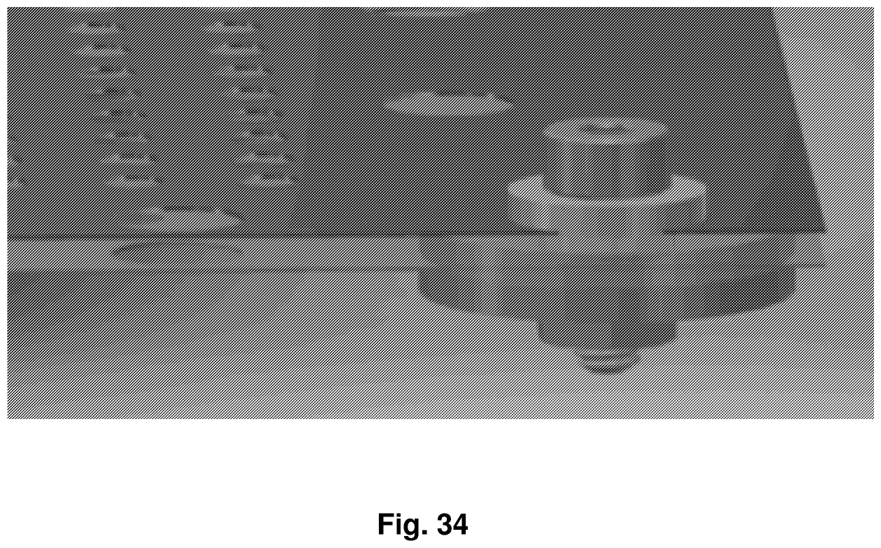

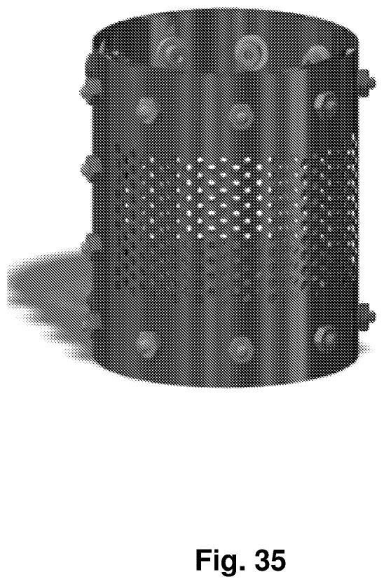

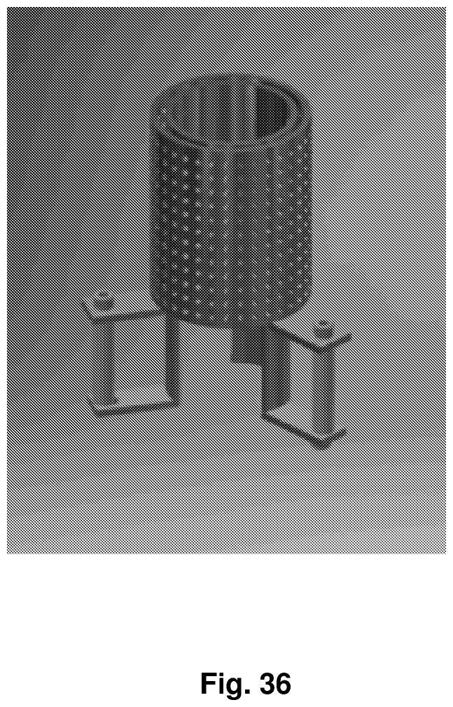

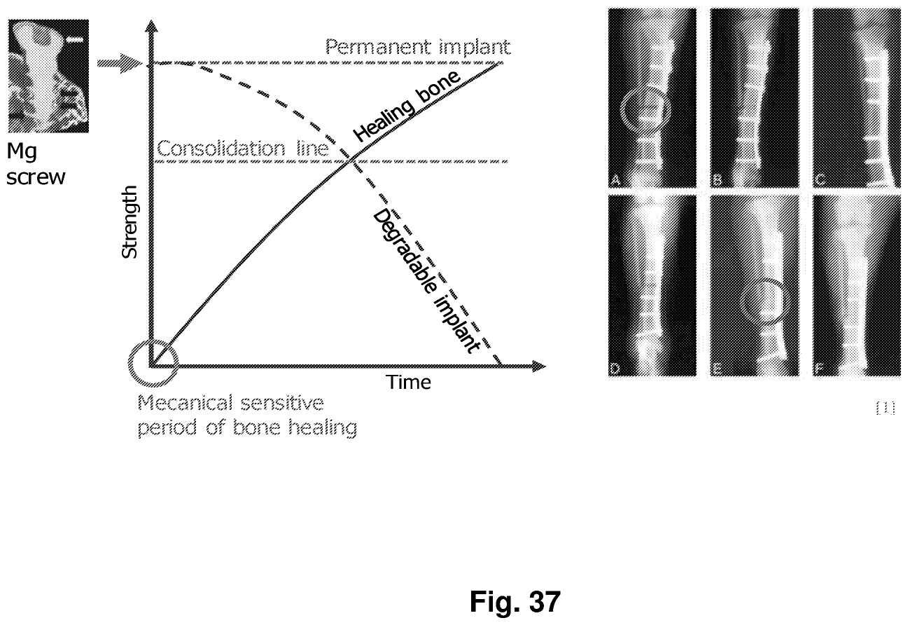

The survey scans for Ti alloy samples treated with a fluence of 1×1017 cgs for Ar irradiation at 60° is displayed in FIG. 28A. The results show the presence of Ti, C, O and N. The Al2p, C1s, N1s, O1s, Ti2p, and V2p are shown in FIG. 28B. The Al2p consist of only one Al peak. On the other hand, C1s is made up of three peaks. Meanwhile N1s is consists of N—O and N—H. There is no change in Ti2p. The O1s is deconvoluted in to 3 peaks. The atomic concentration for control is summarized in Table 7.

### Example 4-3D Complex Geometries of Titanium Dental Implants

Medical devices, which are design to be implanted in living tissues, have to fit perfectly in the tissue defect. There, they will get in contact to host cells and body fluids at the same time, therefore, these biomaterials must have an optimum design of their surface which should promote and facilitate tissue integration1. Among the minimum requirements, biocompatibility and non-toxicity, biomaterials should be developed in a controlled manner in order to promote a reduction of bacteria attachment, a reduction or delay of immune response and enhancement of mesenchymal and osteoblast cells adhesion and differentiation.

During the past few years, the surface modification of dental implants has grown and developed new strategies to enhance the surface properties in order to achieve faster osseointegration and a reduce bacteria attachment. In general terms, the survival rates of dental implants are high, however, there is still implants failure of around 2% and 5% after one month and one year respectively. Implant rejection still occurs and therefore, the main focus of the research society is in the development of new strategies to achieve a suitable surface modification of dental implants which decrease the percentage failure.

Many efforts have been centered on the simulation of the bone matrix chemistry using an active coating with biomacromolecules such collagen or ceramics such hydroxyapatite which supports chemical cues to promotes a proper osseointegration. However, dental implants are going to contact with other tissues, not only hard bone, they have to adjust to soft tissue healing. Regarding dental implants environment, the surface has to induce a faster sealing in soft tissue to avoid bacterial colonization and post infections. The different types of tissue in which medical devices are facing made harder the smart design from a native tissue point of view.

This one reason why surface technology has been developing many strategies in order to create bioactive surfaces which can respond specifically in each context. Plasma-based techniques have shown an attractive method to modify the surface of dental implants developing topography and chemistry changes to respond to different environment. In that sense, the developed features at the nano-scale order can mimic the nanofeatures found in native ECM and interact with cytoplasmatic prolongations such filopodia and lamellipodia in the same order. Surface topography as well physicochemical properties have shown to be key factors of the biological responses affecting fundamental processes, such as the protein adsorption and cell adhesion, proliferation and differentiation.

On the other hand, surface modifications techniques have to take into account the complex structures used in the biomedical field such dental implants or catheters. Many of these surface technologies work in limited conditions using 2D substrates or at small-scale, therefore, some of these technologies are not suitable for the biomedical industry.

In this context, DPNS can produce the surface modification of 3D complex structures and in addition, can be the easy scaleable to the industry level, solving the previous limitations. Furthermore, it achieves nanofeatures in a homogeneous form for the whole surface which will enhance cells interaction and subsequently, implants osseointegration. DPNS becomes a powerful tool capable to effectively modify 3D complex structures as it is shown in the next figures.

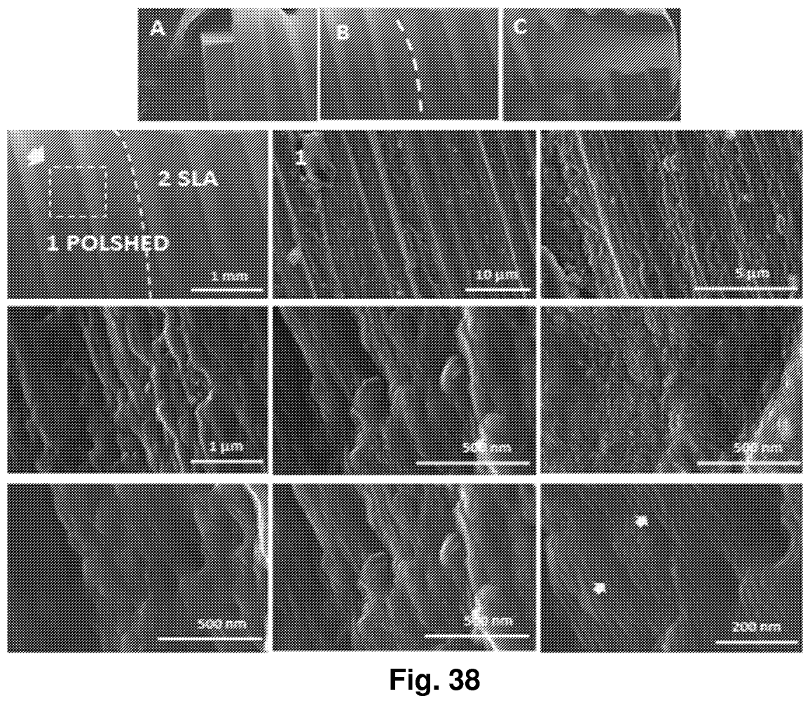

Here, it is presented a clear example of how DPNS can modify any 3D material. As it is observed in FIG. 38 nanofeatures were developed in polished titanium alloy covering the whole implant surface due to the ion bombardment of Argon by DPNS. These homogeneous nanofeatures are presented in similar morphology and size (nanopillars or nanoplatelets of 20 nm).

In these images, it is possible to confirm DPNS as a powerful technology to achieve the modification of complicated geometries with different planes, angles, and topographies.

In addition, these complex structures usually are in the biomedical market with some previous modifications produced by other technologies.

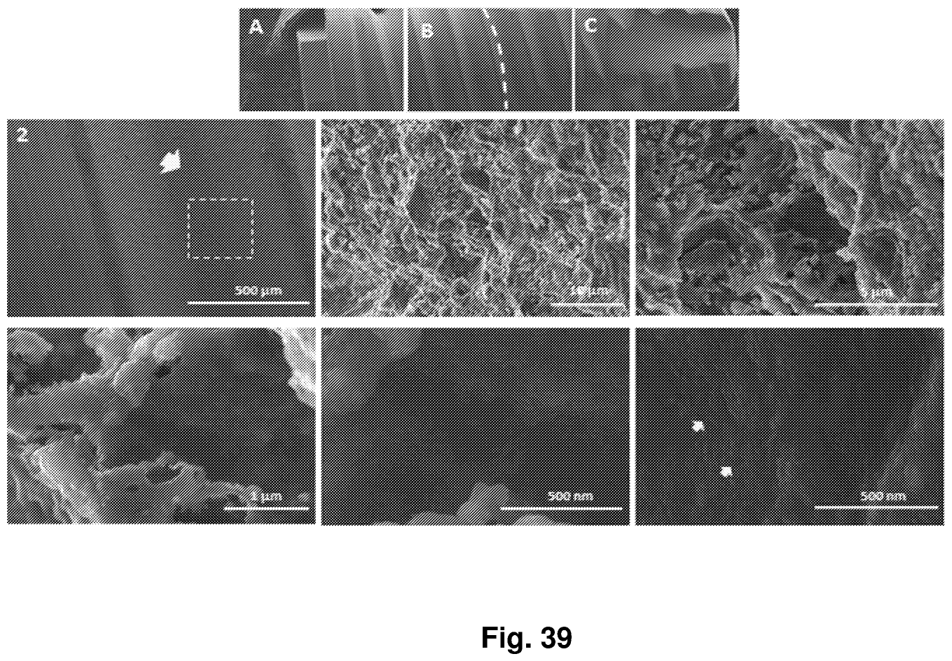

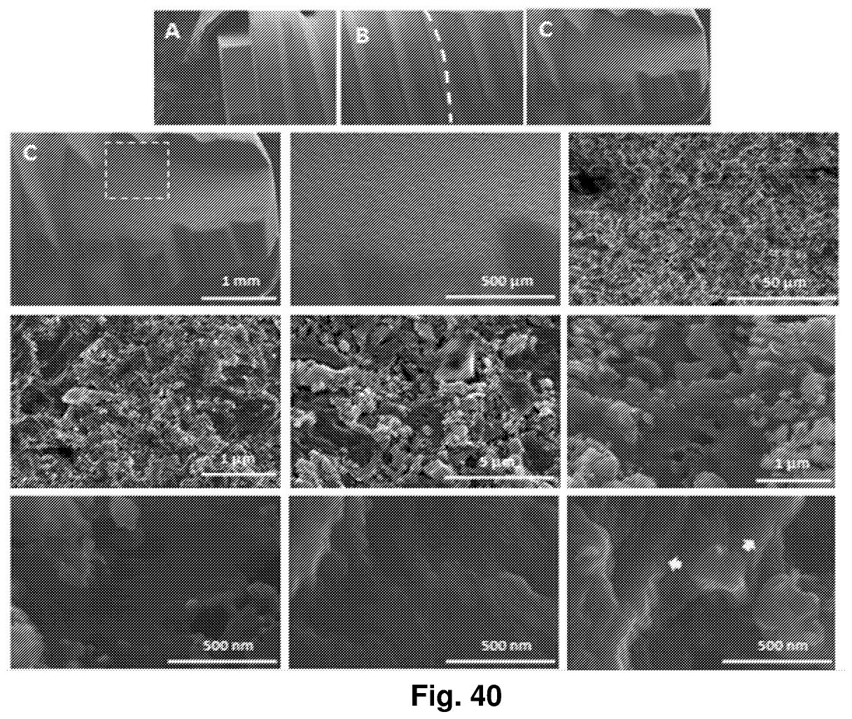

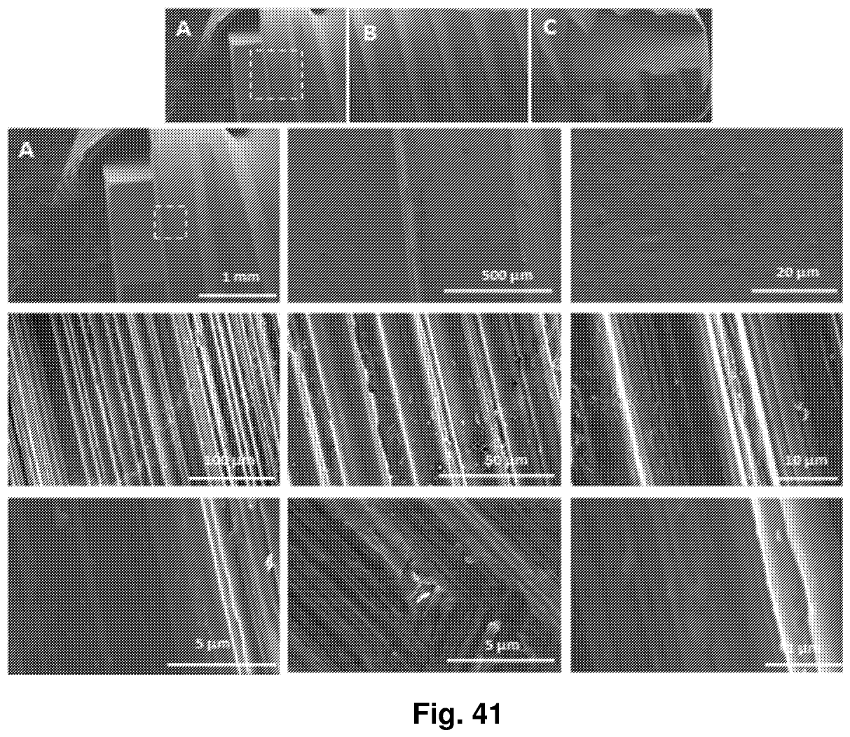

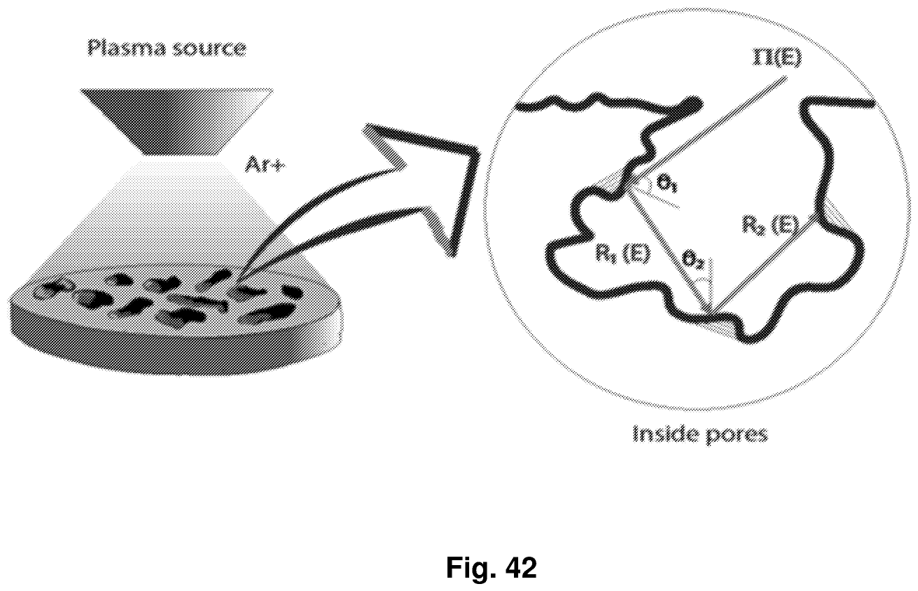

In FIGS. 39 and 40, SEM images revealed a microroughness surface due to the commercial SLA process with new nanofeatures in the middle and lower parts respectively. While the upper part still without any modification (see FIG. 38 polished titanium) which has been shown to reduce bacterial adhesion, the middle and lower parts should increase the osseointegration and long-term fixation. SLA type surface, produced by sandblasting and acid etched process, has shown to promote osteoblast adhesion and differentiation inducing faster osseointegration. The main SLA topography is based on random features at the microscale level, however, using DPNS it has introduced features at the nanoscale level as well. DPNS achieves the surface modification at the nanoscale order of complex devices with other treatments which will improve their osseointegration and increase the success of their clinical application.

### REFERENCES

Smeets, R., Stadlinger, B., Schwarz, F., Beck-Broichsitter, B., Jung, O., Precht, C., . . . & Ebker, T. (2016). Impact of dental implant surface modifications on osseointegration. BioMed Research International, 2016.

Chrcanovic, B. R., Albrektsson, T., & Wennerberg, A. (2014). Reasons for failures of oral implants. Journal of Oral Rehabilitation, 41(6), 443-476.

Ballo, A. M., Omar, O., Xia, W., & Palmquist, A. (2011). Dental implant surfaces—physicochemical properties, biological performance, and trends. In Implant Dentistry—A Rapidly Evolving Practice. InTech.

de Queiroz, J. D. F., de Sousa Leal, A. M., Terada, M., Agnez-Lima, L. F., Costa, I., de Souza Pinto, N. C., & de Medeiros, S. R. B. (2014). Surface modification by argon plasma treatment improves antioxidant defense ability of CHO-k1 cells on titanium surfaces. Toxicology in Vitro, 28(3), 381-387.

Yoshinari, M., Matsuzaka, K., & Inoue, T. (2011). Surface modification by cold-plasma technique for dental implants—Bio-functionalization with binding pharmaceuticals. Japanese Dental Science Review, 47(2), 89-101.

Guastaldi, F. P., Yoo, D., Marin, C., Jimbo, R., Tovar, N., Zanetta-Barbosa, D., & Coelho, P. G. (2013). Plasma treatment maintains surface energy of the implant surface and enhances osseointegration. International journal of biomaterials, 2013.

McNamara, L. E., Sjöstrom, T., Seunarine, K., Meek, R. D., Su, B., & Dalby, M. J. (2014). Investigation of the limits of nanoscale filopodial interactions. Journal of tissue engineering, 5, 2041731414536177.

### Statements Regarding Incorporation by Reference And Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method of generating structures on a substrate, said method comprising:
providing said substrate having a substrate surface;
generating a first directed energetic particle beam from a low temperature plasma source; and
directing said first directed energetic particle beam onto said substrate surface, thereby generating said structures on said surface;
wherein said first directed energetic particle beam has one or more beam properties selected to generate said structures characterized by one or more nanoscale features.

2. A method of generating structures on a substrate, said method comprising:
providing said substrate having a substrate surface;
generating a first directed energetic particle beam and a second directed energetic particle beam from a low temperature plasma source; and
directing said directed energetic particle beams onto said substrate surface, thereby generating said structures on said surface;
wherein said first directed energetic particle beam has one or more first beam properties and said second directed energetic particle beam has one or more second beam properties different from said first beam properties, wherein said first and second beam properties are selected to generate said structures characterized by one or more nanoscale features.

3. The method of claim 1 or 2, wherein said nanoscale features are a preselected nanoscale composition, morphology, topology, topography, crystal structure, density of defects, charge density, bond hybridization, surface energy or any combination thereof.

4. The method of claim 3, wherein said nanoscale morphology is selected from the group consisting of nanorods, nanocones, nanowalls, nanoripples, nanopillars, micropillars, pores, and combinations thereof.

5. The method of claim 3, wherein said nanoscale topography is selected from the group consisting of hexagonal ordered arrays, square ordered arrays, square ordered arrays, and combinations thereof.

6. The method of claim 1 or 2, wherein said nanoscale features correspond to a thermodynamically metastable state.

7. The method of claim 1 or 2, wherein said one or more beam properties is intensity, local fluence, energy, local flux, incident angle, mass, species, cluster size, momentum, charge state, or any combinations thereof.

8. The method of claim 7, wherein said local fluence is between about10¹²-10²⁰ ions/cm², or said energy is between about 50-5000 eV, or said local flux is between about 10¹²-10¹⁶ ions/cm²/s, or said incident angle is between about 0-90°, or said mass is between about 1-131 amu, or said species is selected from the group consisting of H, He, O₂, N₂, Ne, Ar, Kr, Xe, and combinations thereof, or said momentum is between about 10⁻²⁴-10⁻²⁰ kg*m/s, or the charge state is ±1, 2, 3.

9. The method of claim 1 or 2, wherein said first directed energetic particle beam comprises one or more ions, electrons, neutrals, free radicals, or combinations thereof.

10. The method of claim 1 or 2, wherein said one or more beam properties are the ion composition, neutral composition, free radical composition, the ratio of ion abundance to neutral abundance or any combination of these.

11. The method of claim 1 or 2, wherein said first directed energetic particle beam is incident upon said substrate surface from a plurality of directions.

12. The method of claim 11, wherein said plurality of directions of said first directed energetic particle beam is achieved by a porous and non-planar electromagnetic grid in fluid communication with said plasma.

13. The method of claim 12, wherein said porous and non-planar electromagnetic grid is a cylindrical electromagnetic grid.

14. The method of claim 12, wherein said first directed energetic particle beam is incident upon a non-planar inner surface of said substrate.

15. The method of claim 12, wherein said first directed energetic particle beam is incident upon a non-planar outer surface of said substrate.

16. The method of claim 1 further comprising directing one or more additional beams onto said substrate surface, wherein said additional beams are one or more particle beams, radiation beams, directed energetic particle beams, or a combination thereof.

17. The method of claim 16, wherein said one or more additional beams are characterized by at least one beam property that differs from said one or more beam properties of said directed energetic particle beam.

18. The method of claim 16, wherein said one or more additional beams are directed energetic particle beams.

19. The method of claim 16, wherein said one or more additional beams provides a nanoscale feature which is modified as compared to a nanoscale feature which is provided by use of a single beam.

20. The method of claim 1 or 2 further comprising providing one or more additional reactive species or surfactants at a point of contact between said first energetic particle beams and said substrate.

21. The method of claim 1 or 2, wherein said substrate is a metal, a metal oxide, an alloy, a semiconductor, a semi-metal, a non-crystalline metal, a pseudo-alloy, a composite, a ceramic, a polymer, a glass, a tissue, or any combination of these.

22. The method of claim 1 or 2, wherein said substrate surface is the surface of a medical device, an implant, a tissue, a scaffold, a syringe, a needle, a scalpel, a surgical rod, a surgical plate, a surgical screw or any combination of these.

23. The method of claim 1 or 2 further comprising depositing one or more agents on said surface of said substrate.

24. The method of claim 23, wherein said step of depositing said one or more agents is performed by sputtering a target in communication with said first directed energetic particle beam.

25. The method of claim 23, wherein said agents are selected from the group comprising: metals, metal oxides, polymers, glasses, ceramics, tissues, pharmaceuticals, surfactants, and combinations thereof.

26. A system for generating a three dimensional energetic particle beam comprising:
a low temperature plasma source for generating ions; and
at least one porous and non-planar electromagnetic grid in fluid communication with said plasma, wherein said electromagnetic grid accelerates said ions to generate a directed energetic particle beam having one or more selected beam properties.

27. The system of claim 26, wherein said electromagnetic grid has a cylindrical shape with an open top surface, an open bottom surface and a porous axial surface.

28. The system of claim 26 further comprising:
a second porous and non-planar electromagnetic grid in fluid communication with said plasma; and
an agent in communication with said at least one focused ion beam.

29. The system of claim 26, wherein said low temperature plasma source comprises a waveguide operationally connected to a power source, wherein said power source is selected from the group consisting of a dissipated radio frequency, a microwave energy selected from the range of about 10 and 1000 Watts, and a high voltage selected from the range of 100 and 10,000 V, applied to two or more electrodes.

30. The system of claim 26, further comprising a matching network to minimize the reflected power between the power supply and the plasma due to plasma impedance.

31. The system of claim 26, wherein voltages selected from the range of 100 to 5000 V are applied to said at least one porous and non-planar electromagnetic grid to control the absolute and relative ratio fluxes and the acceleration of particles within a directed energetic particle beam.

32. The system of claim 26, wherein said at least one porous and non-planar electromagnetic grid maintains a constant voltage potential during operation of the system.

33. The system of claim 26, wherein said at least one porous and non-planar electromagnetic grid is electrically and physically isolated from the power source.

34. The system of claim 33, wherein said at least one porous and non-planar electromagnetic grids is mounted using ceramic discs, rings, or cylinders.
